SEQUENCE LISTING (28 sequences)

SEQ ID NO: 1 - protein, 650 aa, synthetic construct
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 2 - protein, 215 aa, synthetic construct
EIVLTQSPGTLSLSPGEIATLSCRASQNVRSDLAWYQQKPGQAPRLLIYDASNRATGIPDRFSGSGSGTDFTLTISSLQSEDFAVYYCQQYSNWPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 701 aa, synthetic construct
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSDIQMTQSPSSLSASVGDRVTITCRASQVIRRSLAWYQQKPGKAPKLLIYAASNLASGVPSRFSGSGSGTDFTLTISSLQPEDFATYYCQQSNTSPLTFGQGTKVEIKGQSSRSSGGGGSSGGGGSEVQLVESGGGLVQPGGSLRLSCAASGFSINGSWIFWVRQAPGKGLEWVGAIWPFGGYTHYADSVKGRFTISADTSKNTAYLQMNSLRAEDTAVYYCARWGHSTSPWAMDYWGQGTLVTVSS

SEQ ID NO: 4 - protein, 665 aa, synthetic construct
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 5 - protein, 930 aa, synthetic construct
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 6 - protein, 929 aa, synthetic construct
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHGGGGSGGGGSGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 682 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEIVLTQSPGTLSLSPGEIATLSCRASQNVRSDLAWYQQKPGQAPRLLIYDASNRATGIPDRFSGSGSGTDFTLTISSLQSEDFAVYYCQQYSNWPPLTFGGGTKVEIKGQSSRSSGGGGSSGGGGSDVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSS

SEQ ID NO: 8 - protein, 687 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEIVLTQSPGTLSLSPGEIATLSCRASQNVRSDLAWYQQKPGQAPRLLIYDASNRATGIPDRFSGSGSGTDFTLTISSLQSEDFAVYYCQQYSNWPPLTFGGGTKVEIKGQSSRSSGGGGSSGGGGSDVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSS

SEQ ID NO: 9 - protein, 948 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEIVLTQSPGTLSLSPGEIATLSCRASQNVRSDLAWYQQKPGQAPRLLIYDASNRATGIPDRFSGSGSGTDFTLTISSLQSEDFAVYYCQQYSNWPPLTFGGGTKVEIKGQSSRSSGGGGSSGGGGSDVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSGGGGSGGGGSGGGGSEIVLTQSPGTVSLSPGERVTLSCRASQPIDVYLAWYQQKPGQAPRLLIYGANRRAIDIPDRFSGSGSGTEFTLTINSLQNEDFAVYFCQQYMTWPPVTFGGGTKVEIKGQSSRSSGGGGSSGGGGSDVQLVESGGGVVRPGESLRLSCAASGFSFINFAMTWVRQAPGEGPEWVSLISDDGRGNRPYYADSVKGRFTISRDNSKNILYLQMNSLRVEDTATYYCAKVSWDVFFDYWGLGTVVTVSS

SEQ ID NO: 10 - protein, 948 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEIVLTQSPGTVSLSPGERVTLSCRASQPIDVYLAWYQQKPGQAPRLLIYGANRRAIDIPDRFSGSGSGTEFTLTINSLQNEDFAVYFCQQYMTWPPVTFGGGTKVEIKGQSSRSSGGGGSSGGGGSDVQLVESGGGVVRPGESLRLSCAASGFSFINFAMTWVRQAPGEGPEWVSLISDDGRGNRPYYADSVKGRFTISRDNSKNILYLQMNSLRVEDTATYYCAKVSWDVFFDYWGLGTVVTVSSGGGGSGGGGSGGGGSEIVLTQSPGTLSLSPGEIATLSCRASQNVRSDLAWYQQKPGQAPRLLIYDASNRATGIPDRFSGSGSGTDFTLTISSLQSEDFAVYYCQQYSNWPPLTFGGGTKVEIKGQSSRSSGGGGSSGGGGSDVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSS

SEQ ID NO: 11 - protein, 918 aa, synthetic construct
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGQSSRSSGGGGSSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 12 - protein, 703 aa, synthetic construct
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGKGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGQSSRSSGGGGSSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSS

SEQ ID NO: 13 - protein, 901 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSCSKPEVKKDVKTNSPKHNVNCIYTQTGLSSSPVTVVSSLSYLGSSQLVAPFTHVGSTLAGSNWSVTVPEPFYDKVLCGLAATGGSTSKSSPALPFVSPGKTSASSVTSSVTVLTGLGWYDFFSNWNACYYVATDEATLSNMQLYLTNRSSDRSIIFRGRVSDAYYTNDGVNISSVWELGKGPAQRVWSMGYSDFTFGSATCSLRLSEGPRVVGGGSEVLQVDSGGGGSSGGGGSSRSSQGCEGRNFSKTVPSSLGQHTVECAYVKHKEYDAKSLTLTSSLSYTSDKSDQETVSEQSNGSQLANDVKWQVKAERPYFNNLLCVVSATGSKLQEDSPPFIFVSPAAVTRKIEVKTGGGFTLPPWNSYQQCYYVAFDESQLSSITLTFDTGSGSGSFRDPIGTARNSADYILLRPAQGPKQQYWALDSRVNQSARCSLTAIEGPSLSLTGPSQTLVIE

SEQ ID NO: 14 - protein, 650 aa, synthetic construct
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKKEHVRVFTSNKKTMLGSSAACTYLGQDSRTVGDITLTSLFKKMESGSQTKLDRNVLKKHQHKSSPYEWNFDIGVNLETRATCNLVLKEGVSLEIGHSPSLVVDIITNTQRHTLYNTKYLHGNVTAECTLLGIEKYTANSIIFGKRSDWIIRKGDPILTDLPFKKLTVTINPSTVRCPIVLERGETMHIIEPIESYMEVFPRGTDS

SEQ ID NO: 15 - protein, 445 aa, synthetic construct
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 473 aa, synthetic construct
EIVLTQSPGTLSLSPGEIATLSCRASQNVRSDLAWYQQKPGQAPRLLIYDASNRATGIPDRFSGSGSGTDFTLTISSLQSEDFAVYYCQQYSNWPPLTFGGGTKVEIKRTVAAPSVFIFPPCDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGQSSRSSGGGGSSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSS

SEQ ID NO: 17 - protein, 703 aa, synthetic construct
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGKGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGQSSRSSGGGGSSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSS

SEQ ID NO: 18 - protein, 215 aa, synthetic construct
EIVLTQSPGTLSLSPGEIATLSCRASQNVRSDLAWYQQKPGQAPRLLIYDASNRATGIPDRFSGSGSGTDFTLTISSLQSEDFAVYYCQQYSNWPPLTFGGGTKVEIKRTVAAPSVFIFPPCDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 935 aa, synthetic construct
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 20 - protein, 940 aa, synthetic construct
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 21 - protein, 945 aa, synthetic construct
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 22 - protein, 960 aa, synthetic construct
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 23 - protein, 965 aa, synthetic construct
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 24 - protein, 934 aa, synthetic construct
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHGGGGSGGGGSGGGGSGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 939 aa, synthetic construct
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHGGGGSGGGGSGGGGSGGGGSGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 944 aa, synthetic construct
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHGGGGSGGGGSGGGGSGGGGSGGGGSGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 959 aa, synthetic construct
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHGGGGSGGGGSGGGGSGGGGSGGGGSGGGGSGGGGSGGGGSGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 964 aa, synthetic construct
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHGGGGSGGGGSGGGGSGGGGSGGGGSGGGGSGGGGSGGGGSGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0250343 A1
(43) Pub. Date: Aug. 7, 2025

(54) Antibodies Directed To Tie-2 and VEGF and Methods of Use

(71) Applicant: Unity Biotechnology, Inc., South San Francisco, CA (US)

(72) Inventors: Jason Damiano, South San Francisco, CA (US); Jeff Lu, South San Francisco, CA (US)

(21) Appl. No.: 18/856,780

(22) PCT Filed: Apr. 10, 2023
(86) PCT No.: PCT/US2023/065581

§ 371 Date: Oct. 14, 2024

Related U.S. Application Data
(60) Provisional application No. 63/331,656, filed on Apr. 15, 2022.

(51) Int. Cl. C07K 16/28; A61K 39/00; A61P 9/00; C07K 16/22

(52) U.S. Cl. CPC C07K 16/2863; A61P 9/00; C07K 16/22; A61K 2039/505; C07K 2317/31; C07K 2317/76

(57) ABSTRACT

The invention relates to specific constructs of Tie2/VEGF bispecific antibodies. The invention also relates to pharmaceutical compositions of the bispecific antibodies and methods of using the same for treating a disease in a subject. Also provided are isolated nucleic acids encoding the isolated bispecific antibodies, vectors comprising the isolated nucleic acids, and host cells comprising the vectors.

### CROSS-REFERENCE TO RELATED APPLICATIONS

This application claims the benefit of U.S. Provisional Application No. 63/331,656, filed Apr. 15, 2022, the disclosure of which is incorporated herein by reference in its entirety.

### SEQUENCE LISTING

A Sequence Listing is provided herewith as a Sequence Listing XML, “UNITY-032WO Seq List” created on Mar. 24, 2023, and having a size of 46 KB. The contents of the Sequence Listing XML are incorporated by reference herein in their entirety.

### FIELD OF THE INVENTION

The invention relates to Tie2/VEGF bispecific antibodies and methods of using the same.

### BACKGROUND OF THE INVENTION

Angiogenesis is a tightly regulated process through which new blood vessels form from pre-existing blood vessels. Although angiogenesis is important during development to ensure adequate blood circulation, many disorders are associated with pathological angiogenesis, such as, for example, certain ocular disorders, such as retinopathies. For example, diabetic retinopathy, a microvascular complication of diabetes, occurs in approximately 30% of people with diabetes aged 40 years or older. 3.8% of diabetic adults in this age group have DME, a complication of diabetic retinopathy, which equates to 746,000 people in the 2010 US population (Varma et al. 2014). Diabetic macular edema (DME) is the major cause of visual loss in diabetics, secondary to breakdown of the blood-retinal barrier (BRB) and consequent leakage of plasma and lipid in the macula (Hall 2016; Das, McGuire, and Rangasamy 2015). This leads to swelling of the macula, impairing visual acuity, and ultimately progresses to legal blindness in many patients if left untreated. The management of patients with diabetic retinopathy includes improved hyperglycemic control, the treatment and prophylaxis of cardiovascular complications, regular ophthalmological examinations and medical/surgical ophthalmological interventions. Currently, drug therapies for ocular angiogenesis treatment are based on the administration of anti-VEGF agents via an intravitreal route. The molecules approved for ocular diseases include pegaptanib, ranibizumab and aflibercept.

Despite the efficacy of anti-VEGF therapies in many DR and DME patients, a substantial unmet need still remains as a significant proportion of such patients are refractory to anti-VEGF therapy (Hussain and Ciulla 2016). After 24 weeks of treatment with the most effective regimen (aflibercept 2 mg, every 4 weeks), 32% of patients in the Diabetic Clinical Research Network Protocol T trial showed persistent macular edema by optical coherence tomography (OCT) and central subfield thickness (CST) (Bressler et al. 2018). Twenty percent of eyes that failed to show resolution of macular edema by OCT-CST, and 11% of eyes that did show resolution of macular edema by OCT-CST, failed to achieve 20/40 vision required for automobile driving after 24 weeks of treatment. Thus, there is an unmet medical need for new therapies to treat patients who fail to adequately improve on existing anti-VEGF treatments. The present claimed invention proposes to address this need.

### SUMMARY OF THE INVENTION

The present invention provides bispecific antibodies that specifically bind to both human Tie2 and human VEGF and methods of using the same for therapeutic and diagnostic purposes. The bispecific antibodies of the invention demonstrate unique properties that make them particularly suitable for use in therapy and are distinguished from known standard of care therapies.

Specifically contemplated as part of the disclosed invention is:

Embodiment 1. An isolated bispecific antibody that specifically binds to human Tie2 and to human VEGF selected from the group consisting of:
(a) an IgG heavy chain comprising an amino acid sequence of SEQ ID NO:4; and a light chain kappa comprising an amino acid sequence of SEQ ID NO:2;
(b) an IgG heavy chain comprising an amino acid sequence of SEQ ID NO:3; and a light chain kappa comprising an amino acid sequence of SEQ ID NO:2;
(c) an IgG heavy chain comprising an amino acid sequence of SEQ ID NO:1; and a fully human light chain kappa comprising an amino acid sequence of SEQ ID NO:2;
(d) an IgG heavy chain comprising an amino acid sequence of SEQ ID NO:5; and a fully human light chain kappa comprising an amino acid sequence of SEQ ID NO:2;
(e) an IgG heavy chain comprising an amino acid sequence of SEQ ID NO:6 and a fully human light chain kappa comprising an amino acid sequence of SEQ ID NO:2;
(f) a fully human amino acid sequence of SEQ ID NO:7;
(g) a fully human amino acid sequence of SEQ ID NO:8;
(h) a fully human amino acid sequence of SEQ ID NO:9; and
(i) a fully human amino acid sequence of SEQ ID NO:10.
(j) an IgG heavy chain comprising an amino acid sequence of SEQ ID NO:19; and a light chain kappa comprising an amino acid sequence of SEQ ID NO:2;
(k) an IgG heavy chain comprising an amino acid sequence of SEQ ID NO:20; and a light chain kappa comprising an amino acid sequence of SEQ ID NO:2;
(l) an IgG heavy chain comprising an amino acid sequence of SEQ ID NO:21; and a light chain kappa comprising an amino acid sequence of SEQ ID NO:2;
(m) an IgG heavy chain comprising an amino acid sequence of SEQ ID NO:22; and a light chain kappa comprising an amino acid sequence of SEQ ID NO:2;
(n) an IgG heavy chain comprising an amino acid sequence of SEQ ID NO:23; and a light chain kappa comprising an amino acid sequence of SEQ ID NO:2;
(o) an IgG heavy chain comprising an amino acid sequence of SEQ ID NO:24; and a light chain kappa comprising an amino acid sequence of SEQ ID NO:2;
(p) an IgG heavy chain comprising an amino acid sequence of SEQ ID NO:25; and a light chain kappa comprising an amino acid sequence of SEQ ID NO:2;
(q) an IgG heavy chain comprising an amino acid sequence of SEQ ID NO:26; and a light chain kappa comprising an amino acid sequence of SEQ ID NO:2;
(r) an IgG heavy chain comprising an amino acid sequence of SEQ ID NO:27; and a light chain kappa comprising an amino acid sequence of SEQ ID NO:2; and
(s) an IgG heavy chain comprising an amino acid sequence of SEQ ID NO:28; and a light chain kappa comprising an amino acid sequence of SEQ ID NO:2.
   Embodiment 2. An isolated nucleic acid encoding the isolated bispecific antibody of embodiment 1.
   Embodiment 3. A vector comprising the isolated nucleic acid of embodiment 2. Embodiment A host cell comprising the vector of embodiment 3.
   Embodiment 5. A method of producing an isolated bispecific antibody comprising culturing the host cell of embodiment 4 in a culture medium and isolating the resulting bispecific antibody.
   Embodiment 6. An immunoconjugate comprising the isolated bispecific antibody of embodiment 1.
   Embodiment 7. A fusion polypeptide comprising the isolated bispecific antibody of embodiment 1.
   Embodiment 8. A pharmaceutical composition comprising the isolated bispecific antibody of embodiment 1, the immunoconjugate of embodiment 6, or the fusion polypeptide of embodiment 7.
   Embodiment 9. A method of treating a disease in a subject in need thereof, the method comprising administering to the subject the pharmaceutical composition of embodiment 8.
   Embodiment 10. The method of embodiment 9, wherein the disease comprises ocular diseases, infectious diseases, or ischemic diseases or injuries.
   Embodiment 11. The method of embodiment 10, wherein the infectious diseases comprise sepsis, dengue virus infection, coronavirus infection, tuberculosis, or influenza.
   Embodiment 12. The method of embodiment 10, wherein the ischemic diseases or injuries comprise diabetic nephropathy, acute kidney injury, chronic kidney disease, organ transplantation, or critical limb ischemia.
   Embodiment 13. The method of embodiment 10, wherein the ocular disorders comprise diabetic retinopathy (DR), diabetic macular edema (DME), non-proliferative diabetic retinopathy (nPDR), proliferative diabetic retinopathy (PDR), wet age-related macular degeneration (AMD), dry AMD, geographic atrophy (GA), retinopathy of prematurity (ROP), glaucoma, retinal vein occlusion (RVO), central RVO, or branched RVO.
   Embodiment 14. The isolated bispecific antibody of embodiment 1, or the immunoconjugate of embodiment 6, or the fusion polypeptide of embodiment 7 for use in the treatment of the diseases of embodiments 10-13.
   Embodiment 15. Use of the isolated bispecific antibody of embodiment 1, or the immunoconjugate of embodiment 6, or the fusion polypeptide of embodiment 7 for the manufacture of a medicament for treating the diseases of embodiments 10-13.

### BRIEF DESCRIPTION OF THE DRAWINGS

FIG. 1, panels A-C, show a schematic of some of the bispecific constructs described herein which are based on using a full IgG anti-Tie2 antibody Clone #3 and aflibercept: FIG. 1, panel A, shows Construct #1: an anti-Tie2 full IgG linked to aflibercept in the forward sequence (N-terminal to C-terminal); FIG. 1, panel B, shows Construct #3: an anti-Tie2 full IgG linked to a GS linker where n=4 linked to aflibercept in the forward sequence; FIG. 1, panel C, shows Construct #13: an anti-Tie2 full IgG linked aflibercept, but in the reverse sequence (C-terminal to N-terminal). See also FIG. 7 and Table 1.

FIG. 2, panels A-D, show a schematic of some of the bispecific constructs described herein which are based on using a full IgG anti-Tie2 antibody Clone #3 and either the B20 VEGF binder or ranibizumab: FIG. 2, panel A, shows Construct #2: an anti-Tie2 full IgG linked to a VEGF scFv clone B20; FIG. 2, panel B, shows Construct #10: an anti-Tie2 full IgG linked to a ranibizumab Fab fragment; FIG. 2, panel C, shows Construct #8: an anti-Tie2 full IgG linked to a ranibizumab Fab fragment using a long linker; FIG. 2, panel D, shows Construct #9: similar to Construct #8, but where the variable heavy and variable light chains of the ranibizumab Fab fragment are reversed. See also FIGS. 8A-8B and Table 1.

FIG. 3, panels A-D, show a schematic of some of the bispecific constructs described herein which are based on using aflibercept and antibody fragments of anti-Tie2 antibody Clones #3 and #2: FIG. 3, panel A, shows Construct #5: aflibercept linked to an scFv of anti-Tie2 Clone #3; FIG. 3, panel B, shows Construct #6: aflibercept linked to a Fab fragment of anti-Tie2 Clone #3; FIG. 3, panel C, shows Construct #7: aflibercept linked to an scFv of anti-Tie2 Clone #2, which is further linked to an scFv of anti-Tie2 Clone #3; FIG. 3, panel D, shows Construct #12: aflibercept linked to an scFv of anti-Tie2 Clone #3, which is further linked to an scFv of anti-Tie2 Clone #2. See also FIG. 7 and Table 1.

FIG. 4, panels A-B, show Akt activation as measured by EC50 in nM of certain bispecific antibody constructs (Constructs #1 and #5 and Ang2/VEGF) as compared to controls (ANG1, anti-Tie2 Clone #3) (FIG. 4, panel A). The Akt potencies were quantified as EC50 values in nM (FIG. 4, panel B). See Example 1.

FIG. 5, panels A-B, show the VEGF neutralization in vitro potency, as measured by % VEGF activity (FIG. 5, panel A) and shows the phosphor-Tie2 (pTIE2) in vitro potency, as measured by the % Ang1 activation (FIG. 5, panel B) of Constructs #2, #4 and #5 as compared to Ang1, Anti-Tie2 and aflibercept-LALA-PG. See Examples 2 and 3.

FIG. 6, panels A-E, show the in vitro phospho-Tie2 potency, as measured by the % Ang1 activation of two bispecifics of the invention, Constructs #1 (FIG. 6, panel C) and #5 (FIG. 6, panel D) with and without the addition of VEGF, as compared to an Ang1 control (FIG. 6, panel A) and an anti-Tie2 Clone #3 (FIG. 6 panel B). The results are quantified as a p-Tie2 EC50 nM with or without VEGF (FIG. 6 panel E). See Example 4.

FIG. 7 shows a table summary comparing certain Tie2-VEGF Trap bispecific constructs of the invention by the format of the Tie2 binder and the format of the VEGF binder, the CMC characteristics of yield (in mg), concentration [conc] in mg/ml, purity SEC %, and stress stability (37° C. at 2 weeks), as well as the in vitro potencies of each bispecific antibody construct tested in terms of the phospho-Tie2 activating and VEGF neutralizing in vitro potencies as measured by EC50 (in nM). N/A=data is unmeasurable, TBD=data is pending, N.D.=Not Done. See FIGS. 1A-C, 3A-D, Table 1 and Examples 2, 3 and 5.

FIGS. 8A-8B both show a table summary comparing certain Tie2-anti-VEGF monoclonal antibody bispecific constructs of the invention by the format of the Tie2 binder and the format of the VEGF binder, the CMC characteristics of yield (in mg), concentration [conc] in mg/ml, purity SEC %, and stress stability (37° C. at 2 weeks), as well as the in vitro potencies of each bispecific antibody construct in terms of the phospho-Tie2 activating and VEGF neutralizing in vitro potencies as measured by EC50 (in nM). N/A=data is unmeasurable, TBD=data is pending, N.D.=Not Done. See FIG. 2, panels A-D, Table 1 and Examples 2, 3 and 5.

FIG. 9, panels A-B, show the VEGF neutralization in vitro potency, as measured by % inhibition of VEGF activity (FIG. 9, panel A) and shows the phosphor-Tie2 (pTIE2) in vitro potency, as measured by the % Ang1 activation (FIG. 9, panel B) of Constructs #3 and #1 as compared to Ang1, Anti-Tie2 Clone #3 and aflibercept. See Examples 2 and 3. FIG. 9, panel C, shows a table of EC50 values (pTIE2 EC50 (nM) and VEGF EC50 (nM)) for various constructs/controls.

FIG. 10, panels A-D, show the in vitro phospho-Tie2 potency, as measured by the % Ang1 activation of two bispecifics of the invention, Constructs #1 (FIG. 10, panel B) and #3 (FIG. 10, panel C) with and without the addition of VEGF, as compared to an anti-Tie2 Clone #3 control (FIG. 10, panel A). The results are quantified as a p-Tie2 EC50 nM with or without VEGF (FIG. 10, panel D). See Example 4.

FIG. 11, panels A-B, show Akt activation as measured by EC50 in nM of certain bispecific antibody constructs (Constructs #1 and #3) as compared to control (ANG1) (FIG. 11, panel A and FIG. 11, panel B). The Akt potencies were quantified as EC50 values in nM (FIG. 11, panel B). See Example 1.

FIG. 12 shows ERK activation as measured by western blot by bispecific Construct #3 as compared to control (ANG1) (FIG. 12). See Example 9.

FIG. 13, panels A-C, show target engagement of phospho-Tie2 in naïve choroid tissue of young mice using the bispecific Construct #3 or control (NSigG) at 2.5 ug/eye, or 5 ug/eye or 10 ug/eye at certain timepoints: 2 h (FIG. 13, panel B) or 7d (FIG. 13, panel C). See Example 8.

FIG. 14, panels A-C, show target engagement of phospho-Tie2 in naïve retinal tissue of young mice using the bispecific Construct #3 or control (NSigG) at 2.5 ug/eye, or 5 ug/eye or 10 ug/eye at timepoints: 2 h (FIG. 14, panel B) or 7d (FIG. 14, panel C). See Example 8.

FIG. 15, panels A-B, show in vivo efficacy studies for avascular (FIG. 15, panel A) and neovascularization (FIG. 15, panel B) in the OIR murine disease model comparing bispecific Construct #3 at 5 ug and 20 ug against a murine Ang2/VEGF antibody (FMS) at 5 ug and 20 ug, aflibercept-LALA-PG at 20 ug, Eyelea® at 20 ug, and an irrelevant negative control NS-huIgG1. See Example 10 and Tables 3 and 4.

FIG. 16, panels A-B, show target engagement of pTie2 (FIG. 16, panel A) and Ang2 (FIG. 16, panel B) in the hyperoxic retina when dosed at P13 and harvested at P15 using Constructs #1, #3 compared with a murine Ang2/VEGF antibody (FMS) all against an irrelevant negative control NSIgG. For FIG. 16, panel A, each point represents the average amount of protein detected and error bars represent SD. For FIG. 16, panel B, each bar represents the average amount of protein detected and error bars represent SD. See Example 6.

FIG. 17, panels A-B, show target engagement of pTie2 (FIG. 17, panel A) and Ang2 (FIG. 17, panel B) in the hyperoxic retina when dosed at P15 and harvested at P16 using Constructs #1, #3 compared with a murine Ang2/VEGF antibody (FMS) all against an irrelevant negative control NSIgG. For FIG. 17, panel A, each point represents the average amount of protein detected and error bars represent SD. For FIG. 17, panel B, each bar represents the average amount of protein detected and error bars represent SD. See Example 6.

### DETAILED DESCRIPTION OF THE INVENTION

### I. Definitions

“Tie2” is also known as angiopoietin-1 receptor, or TEK receptor tyrosine kinase, or CD202B (cluster of differentiation 202B), and is a protein that in humans is encoded by the TEK gene (Partanen J et al., (April 1992). A novel endothelial cell surface receptor tyrosine kinase with extracellular epidermal growth factor homology domains. Molecular and Cellular Biology. 12 (4): 1698-707). This receptor possesses a unique extracellular domain containing three immunoglobulin-like loops, three epidermal growth factor-like repeats and three fibronectin type Ill-like repeats (see Fiedler et al., 2006. Angiopoietins: A Link between Angiogenesis and Inflammation. Trends in Immunology 27 (12): 552-58; Barton et al., Crystal structures of the Tie2 receptor ectodomain and the angiopoietin-2-Tie2 complex. Nature Struc. & Mol. Biology, 13, pp 524-532 (2006)). The contact residues for angiopoietin-1 and angiopoietin-2 are mostly overlapping on the Tie-2 receptor and are predominantly located in the second Ig-like loop, as suggested by the analysis of the crystal structure of the Ang2/Tie2 complex (Barton et al., Nat Str Biol 2006). Other work supports the concept that the binding domains for Ang1 and Ang2 are similar or identical (Fiedler et al., Angiopoietin-1 and angiopoietin-2 share the same binding domains in the Tie-2 receptor involving the first Ig-like loop and the epidermal growth factor-like repeats. JBC. Vol. 278 (3): 1721-7 (2003)). The amino acid sequence of an exemplary human Tie2 may be found under UniProt Accession Number Q02763.

“Vascular endothelial growth factor (VEGF)” is a signal protein produced by many cells that stimulates the formation of blood vessels. VEGF belongs to a sub-family of growth factors, the platelet-derived growth factor family of cystine-knot growth factors. VEGF's normal function is to create new blood vessels during embryonic development, new blood vessels after injury, muscle following exercise, and new vessels (collateral circulation) to bypass blocked vessels. Aberrant VEGF expression can also contribute to disease. Solid cancers cannot grow beyond a limited size without an adequate blood supply, cancers that can express VEGF are able to grow and metastasize. Overexpression of VEGF can cause vascular disease in the retina of the eye and in other areas of the human body, such as, for example, in the kidney. As a result, VEGF is a clinically validated driver of angiogenesis and neutralization of VEGF by, for example, using an anti-VEGF blocking antibody, can be used to treat disorders associated with pathological angiogenesis. The amino acid sequence of an exemplary human VEGF may be found under UniProtKB Accession Number A0A024RD33.

The term “about” as used herein refers to the usual error range for the respective value readily known to the skilled person in this technical field. Reference to “about” a value or parameter herein includes (and describes) embodiments that are directed to that value or parameter per se.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Active” or “activity” or “biological activity” in the context of a bispecific antibody of the present invention is the ability to both (a) agonize (partially or fully activate) a biological activity of human Tie2 in vitro and/or in vivo as well as the ability to (b) antagonize (partially or fully inhibit) a biological activity of human VEGF.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein.

An “affinity matured” antibody refers to an antibody with one or more alterations in one or more hypervariable regions (CDRs) and/or framework regions (FRs), compared to a parent antibody which does not possess such alterations, such alterations resulting in an improvement in the affinity of the antibody for antigen.

“Allosteric activation of Tie2” is the activation of Tie2 by an agonistic anti-Tie2 antibody that specifically interacts with regions of Tie2 outside of the described ligand binding or active site, such that the binding results in a change in Tie2 conformation or clustering that enhances the receptor's activity.

The term “antibody” herein is used in the broadest sense and encompasses various antibody structures, including but not limited to monoclonal antibodies, polyclonal antibodies, bispecific antibodies, and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and bispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire light (L) chain along with the variable region domain of the heavy (H) chain (VH), and the first constant domain of one heavy chain (CH1). Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md. (1991).

“Fv” consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

“Single-chain Fv” also abbreviated as “sFv” or “scFv” are antibody fragments that comprise the VH and VL antibody domains connected into a single polypeptide chain. The sFv polypeptide may further comprise a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing sFv fragments (see preceding paragraph) with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al., Proc. Natl. Acad. Sci. USA, 90:6444-6448, 1993.

A “blocking” antibody or an “antagonist” antibody is one which inhibits or reduces biological activity of the antigen it binds. Certain blocking antibodies or antagonist antibodies substantially or completely inhibit the biological activity of the antigen.

An “agonistic” or “activating” antibody is one which activates, stimulates or increases biological or signaling activity of the antigen it binds. In some situations, it is contemplated that an agonistic antibody can act in a similar manner to how a ligand engages and activates its cognate receptor. In other situations, it is contemplated that the bispecific antibodies of the invention are considered agonistic if they induce Tie2 signaling as determined by increased levels of one or more of intracellular phosphorylated Tie2 (pTie2), and/or phosphorylated Akt (pAkt), and/or phosphorylated ERK (pERK). In addition, it is further contemplated that an agonistic Tie2 antibody of the invention can also be capable of activating downstream signaling of its target antigen in the presence or absence of endogenous activating (i.e. Ang1) or inhibitory (i.e. Ang2) ligands. See Damiano J., et al., WO/2021/102173 for examples of assays used to determine this.

The term “biparatopic” as used herein, refers to a bispecific antibody where the first antigen-binding moiety and the second antigen-binding moiety bind to different epitopes on the same antigen.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ and μ, respectively.

“Complement factors” means the various proteins and glycoproteins that make up the complement cascade which is a part of the immune system that enhances (complements) the ability of antibodies and phagocytic cells to clear microbes and damaged cells from an organism, promote inflammation, and attack the pathogen's cell membrane. It is part of the innate immune system. Complement factors contemplated herein include, for example, C1, C2, C2a, C2b, C3, C3a, C3b, C4, C4a, C4b, C5, C5a, C5b, C6, C7, C8 and C9.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down-regulation of cell surface receptors (e.g., B cell receptor); and B cell activation.

“Framework” or “framework region” or “FR” refers to variable domain residues other than hypervariable region (CDR) residues. The FR of a variable domain generally consists of four FR domains: FR1, FR2, FR3, and FR4.

The terms “full-length antibody,” “intact antibody,” and “whole antibody” are used herein interchangeably to refer to an antibody having a structure substantially similar to a native antibody structure or having heavy chains that contain an Fc region as defined herein.

The term “fusion polypeptide” encompasses bispecific antibodies of the invention fused to, for example, an immunoglobulin Fc region. An Fc region may comprise, for example, a CH3 domain of an immunoglobulin, which may be naturally occurring or modified in some way. Such Fc fusion polypeptides can exhibit a greater half-life in vivo than the unfused counterpart. Also, a fusion to an Fc region allows for dimerization/multimerization of the fusion polypeptide. As contemplated, the Fc region may be a naturally occurring Fc region, or may be altered to improve certain qualities, such as therapeutic qualities, circulation time, decrease aggregation problems, for example. In another embodiment, a fusion polypeptide contemplates bispecific antibody fragments fused to a ligand, such as, for example, Ang1 to enhance agonistic activity of the fusion polypeptide. In another embodiment, a fusion polypeptide contemplates an bispecific antibody fragments fused to, for example, a cytokine to elicit other desired biology.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, FR residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr Op. Struct. Biol. 2:593-596 (1992).

The term “variable” refers to the fact that certain segments of the variable domains differ extensively in sequence among antibodies. The variable or “V” domain mediates antigen binding and defines specificity of a particular antibody for its particular antigen. However, the variability is not evenly distributed across the span of the variable domains. Instead, the V regions consist of relatively invariant stretches called framework regions (FRs) of 15-30 amino acids separated by shorter regions of extreme variability called “hypervariable regions” that are each 9-12 amino acids long. The term “hypervariable region” or “CDR” when used herein refers to the amino acid residues of an antibody which are responsible for antigen-binding. The hypervariable region generally comprises amino acid residues from, for example, around about residues 24-34 (L1), 50-56 (L2) and 89-97 (L3) in the VL, and around about residues 26-35 (H1), 49-65 (H2) and 95-102 (H3) in the VH (in one embodiment, H1 is around about residues 31-35); Kabat et al., Sequences of Proteins of Immunological Interest, 5th Ed. Public Health Service, National Institutes of Health, Bethesda, Md. (1991)) and/or those residues from a “hypervariable loop” (e.g., residues 26-32 (L1), 50-52 (L2), and 91-96 (L3) in the VL, and 26-32 (H1), 53-55 (H2), and 96-101 (H3) in the VH; Chothia and Lesk, J. Mol. Biol. 196:901-917 (1987). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). Accordingly, the CDR and FR sequences generally appear in the following sequence in VH (or VL): FR1-H1 (L1)-FR2-H2(L2)-FR3-H3(L3)-FR4. The constant domains are not involved directly in binding an antibody to an antigen, but exhibit various effector functions, such as participation of the antibody in antibody dependent cellular cytotoxicity (ADCC).

The terms “residue numbering as in Kabat,” “Kabat amino acid residue,” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g. residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g, Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system. Unless otherwise indicated, CDR residues and other residues in the variable domain (e.g., FR residues) are numbered herein according to Kabat et al., supra.

An “immunoconjugate” is an antibody conjugated to one or more heterologous molecule(s) that delivers a cell-killing or cell-altering activity, including but not limited to a small molecule drug (inhibitor or activator), or cytotoxic agent, such as chemotherapeutic agents or drugs, growth inhibitory agents, toxins (e.g., protein toxins, enzymatically active toxins of bacterial, fungal, plant, or animal origin, or fragments thereof), or radioactive isotopes.

The term an “isolated bispecific antibody” when used to describe the various bispecific antibodies disclosed herein, means a bispecific antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In some embodiments, a bispecific antibody of the invention is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) approaches. For a review of methods for assessment of antibody purity, see, for example, Flatman et al., J. Chromatogr. B 848:79-87 (2007). In certain embodiments, the bispecific antibody of the invention will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or silver stain. Isolated bispecific antibody of the invention includes bispecific antibodies in situ within recombinant cells, because at least one component of the bispecific antibody of the invention's natural environment will not be present. Ordinarily an isolated bispecific antibody of the invention will be prepared by at least one purification step.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “bispecific antibody” is used in the broadest sense and specifically covers an antibody or antibody fragment having polyepitopic specificity (i.e., is capable of binding to two different epitopes on one biological molecule or each epitope on a different biological molecule). Such bispecific antibodies include, but are not limited to, full-length antibodies, antibodies having two or more VL and VH domains, antibody fragments such as Fab, Fv, dsFv, scFv, diabodies, bispecific diabodies and triabodies, antibody fragments that have been linked covalently or non-covalently. “Polyepitopic specificity” refers to the ability to specifically bind to two or more different epitopes on the same or different target(s). According to one embodiment, the bispecific antibody of the invention in an IgG1 form binds to each epitope with an affinity of 5 μM to 0.001 μM, 3 μM to 0.001 μM, 1 μM to 0.001 μM, 0.5 μM to 0.001 μM or 0.1 μM to 0.001 μM.

The term “linker” as used herein refers to fusion protein linkers, which may be, for example, flexible linkers, well known in the art. (see, e.g., Hollinger P, et al., PNAS USA. 90:6444-6448 (1993); Poljak RJ. Structure 2: 1121-1123 (1994)). Commonly used flexible linkers have sequences consisting of stretches of Gly and Ser residues (“GS” or “G4S” linker). An example of such a GS flexible linker has the sequence of (Gly-Gly-Gly-Gly-Ser)-n. By adjusting the copy number “n”, the length of this GS linker can be optimized to achieve appropriate separation of the functional domains, or to maintain necessary inter-domain interactions, such as, for example, in the construct of the bispecific antibodies of the invention, a VH and VL domain, two scFv antibody fragments or a variable domain and an extracellular trap protein or a scFv antibody fragment and an extracellular trap protein. Other examples of suitable, non-immunogenic linker peptides are: (SG4)n or G4(SG4)n or GQSSRSS(G4S)n flexible peptide linkers, or rigid/non-flexible linkers (EAAAK)n or (XP)n, in both cases where “n” is a number between 1 and 10, or between 1 and 4, as well as oligomers of such linkers.

With regard to the binding of a bispecific antibody of the invention to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a bispecific antibody of the invention having a KD for the target of 10⁻⁴ M or lower, alternatively 10⁻⁵ M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a KD in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and KD values are inversely related. A high affinity for an antigen is measured by a low KD value. In one embodiment, the term “specific binding” refers to binding where a molecule, such as a bispecific antibody of the invention, binds to a particular polypeptide or epitope on a particular polypeptide without substantially binding to any other polypeptide or polypeptide epitope.

The term “Tie2 non-ligand competitive binder” is a bispecific antibody of the invention of the invention that does not compete for the active site of Tie2 with either Ang 1 or Ang 2, while still allowing either Ang 1 or Ang 2 to bind at the active site.

A “nucleic acid encoding a bispecific antibody of the invention” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, “administering” is meant a method of giving a dosage of a bispecific antibody of the invention or a composition such as a pharmaceutical composition including a bispecific antibody of the invention to a subject in need thereof. Such compositions utilized in the treatment methods described herein can be administered, for example, locally to the tissues or organs affected by disease. In one embodiment, such administration is injection of the compositions of the invention directly in the affected eye, for example, intravitreally, ocularly, intraocularly, subretinally, or suprachoroidally and the like. In another embodiment, compositions utilized in the treatment methods described herein can be administered, for example, sub-cutaneously near the diseased tissues or organs.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. A “subject” may be a “patient.”

As used herein, “treatment” (and “treat” or “treating”) refers to clinical intervention in an attempt to alter the natural course of the individual being treated and can be performed either for prophylaxis or during the course of clinical pathology. Desirable effects of treatment include, but are not limited to, preventing occurrence or recurrence of a disease, alleviation of symptoms of such disease, diminishment of any direct or indirect pathological consequences of a disease, decreasing the rate of disease progression, amelioration or palliation of a disease state, and remission or improved prognosis. In some embodiments, bispecific antibodies of the invention are used to delay development of a disease or to slow the progression of a disease.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

A “mutation” is a deletion, insertion, or substitution of a nucleotide(s) relative to a reference nucleotide sequence, such as a wild-type sequence.

A “variant” or “mutant” of a starting or reference bispecific antibody (e.g., a reference antibody or its variable domain(s)/CDR(s)), is a polypeptide that (1) has an amino acid sequence different from that of the starting or reference bispecific antibody and (2) was derived from the starting or reference bispecific antibody through either natural or artificial (man-made) mutagenesis. Such variants include, for example, deletions from, and/or insertions into and/or substitutions of, residues within the amino acid sequence of the bispecific antibody of interest, referred to herein as “amino acid residue alterations.” Thus, a variant CDR refers to a CDR comprising a variant sequence with respect to a starting or reference bispecific antibody sequence (such as that of a source antibody or antigen binding fragment). An amino acid residue alteration, in this context, refers to an amino acid different from the amino acid at the corresponding position in a starting or reference bispecific antibody sequence (such as that of a reference antibody or fragment thereof). Any combination of deletion, insertion, and substitution may be made to arrive at the final variant or mutant construct, provided that the final construct possesses the desired functional characteristics. The amino acid changes also may alter post-translational processes of the bispecific antibody, such as changing the number or position of glycosylation sites.

A “wild-type (WT)” or “reference” sequence or the sequence of a “wild-type” or “reference” antibody, such as an CDR or a variable domain of a reference antibody, may be the reference sequence from which variant antibodies are derived through the introduction of mutations. In general, the “wild-type” sequence for a given antibody is the sequence that is most common in nature. Similarly, a “wild-type” gene sequence is the sequence for that gene which is most commonly found in nature. Mutations may be introduced into a “wild-type” gene (and thus the protein it encodes) either through natural processes or through man-induced means. The products of such processes are “variant” or “mutant” forms of the original “wild-type” protein or gene.

A “reference antibody,” as used herein, refers to an antibody or fragment thereof whose antigen-binding sequence serves as the template sequence upon which diversification according to the criteria described herein is performed. An antigen-binding sequence generally includes an antibody variable region, at least one CDR, including framework regions.

### Compositions and Methods

The invention provides novel bispecific antibodies that bind to Tie2 and to VEGF, and methods of making and using the same, for example, for therapeutic and diagnostic uses. Bispecific antibodies of the invention are useful, e.g., for the diagnosis or treatment of various disorders, described herein.

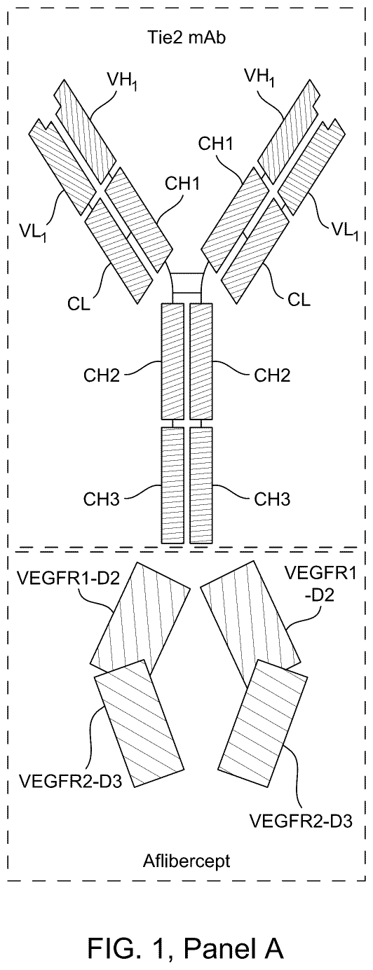

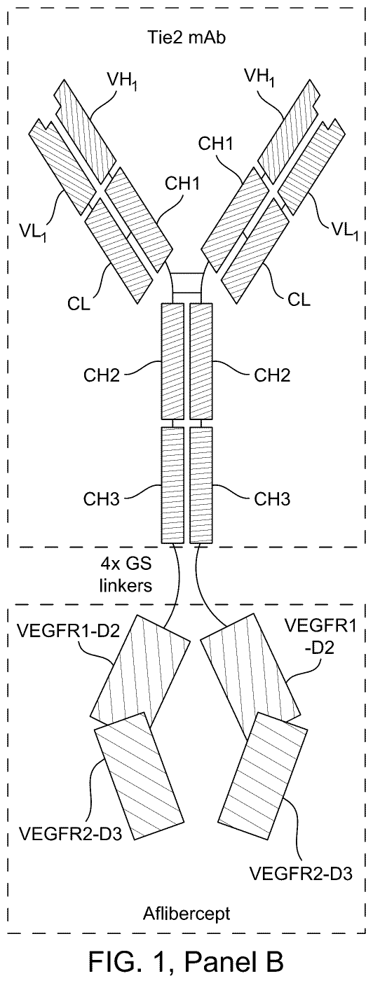

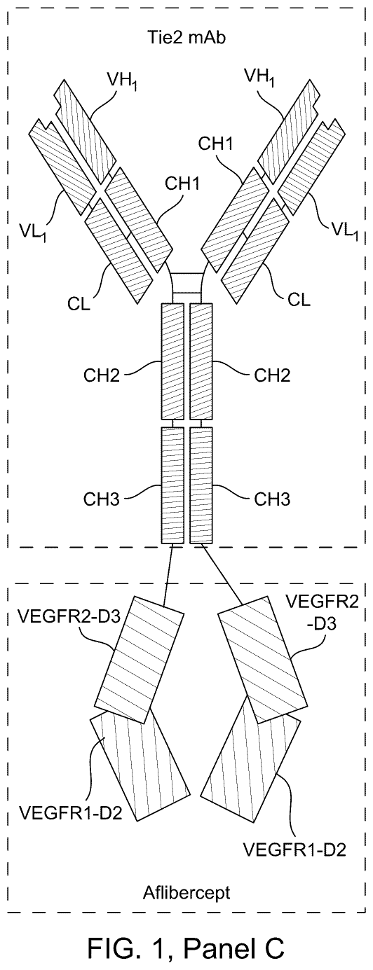

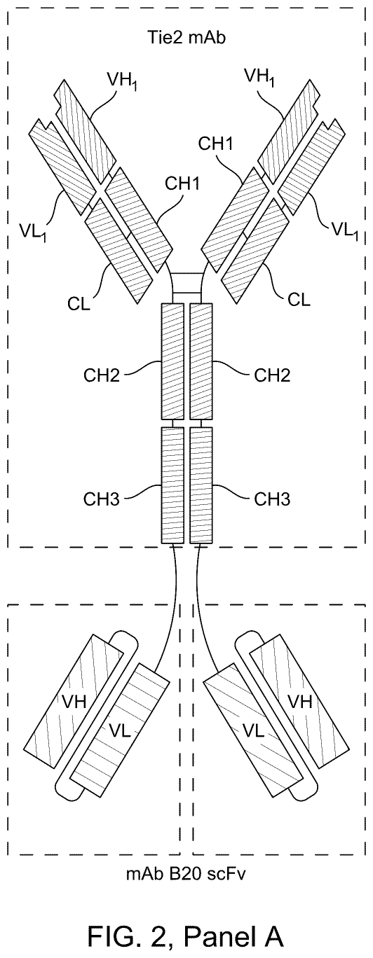

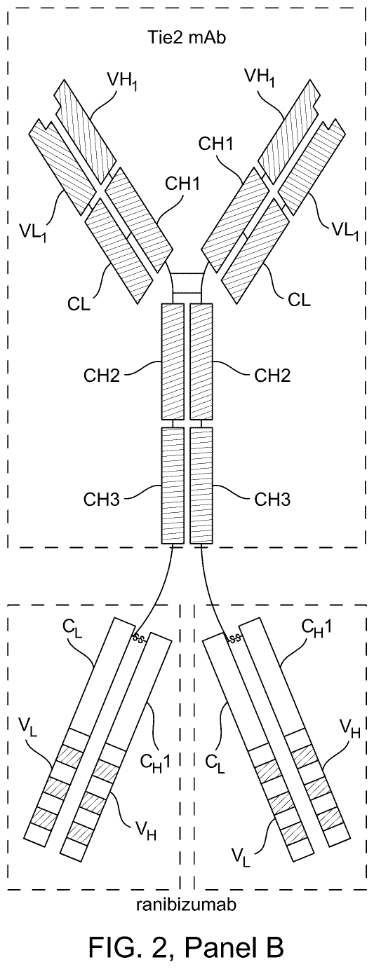

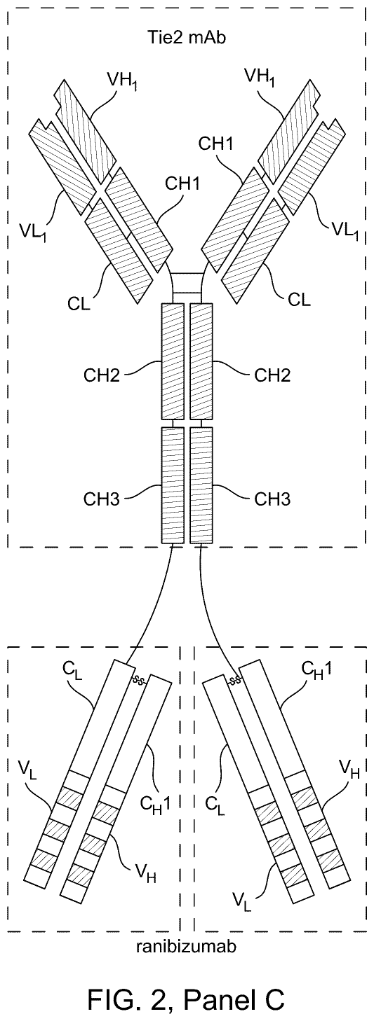

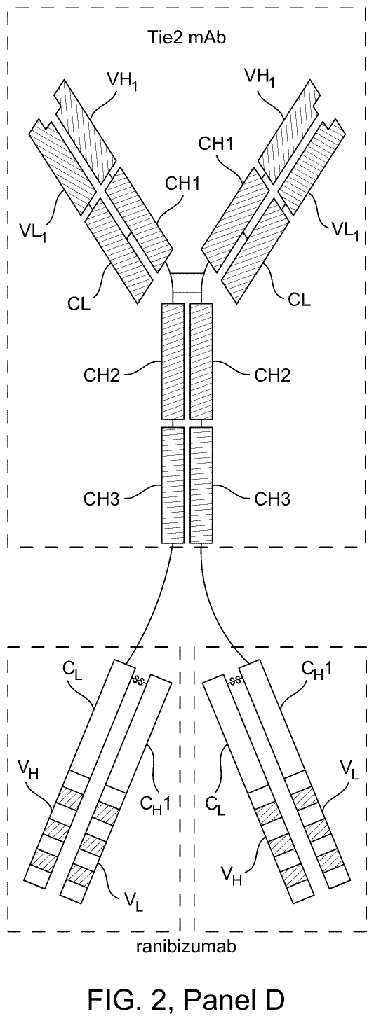

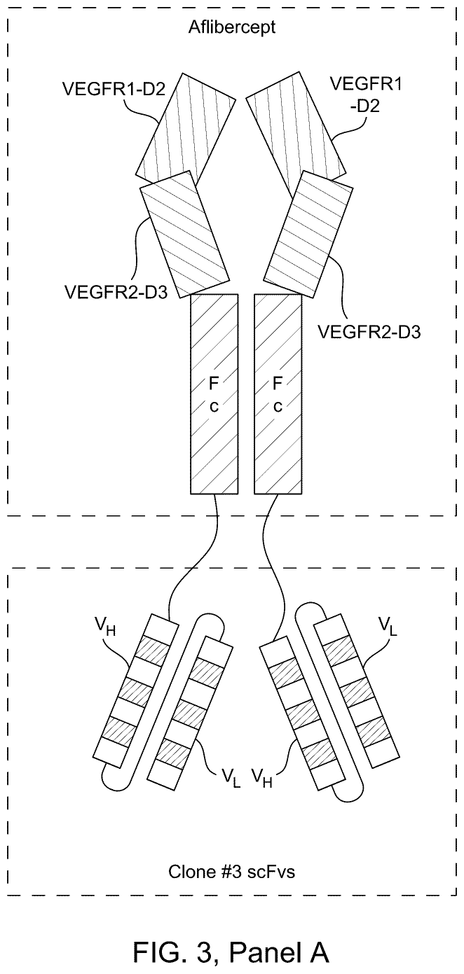

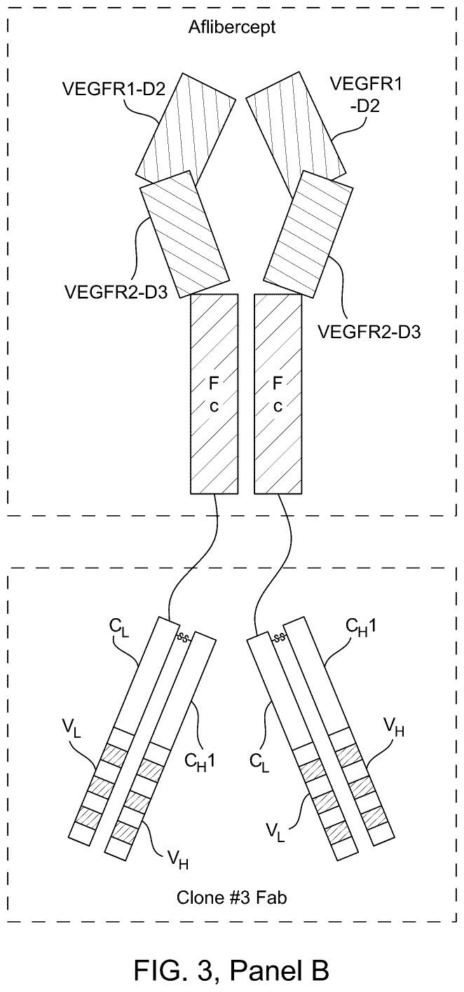

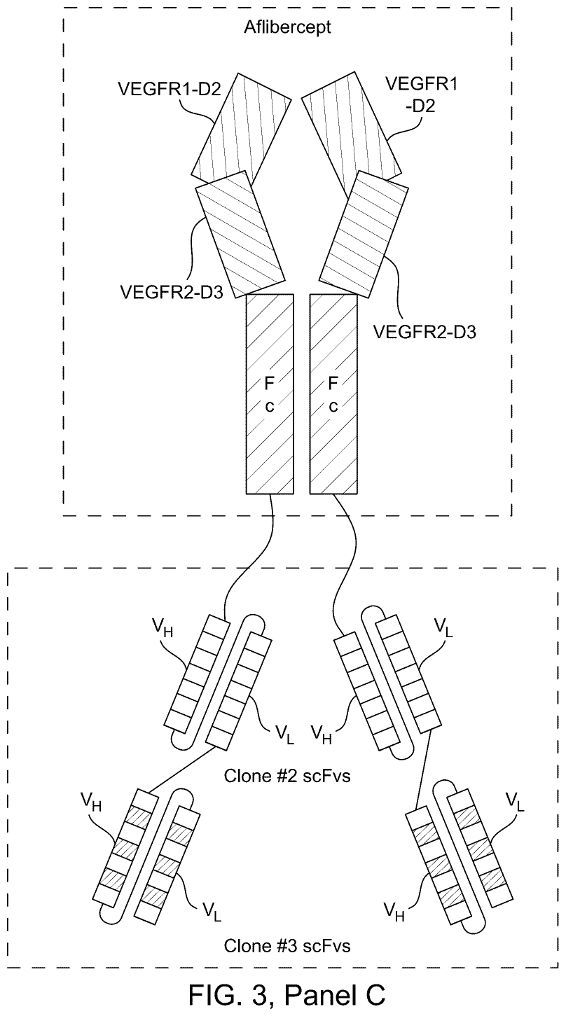

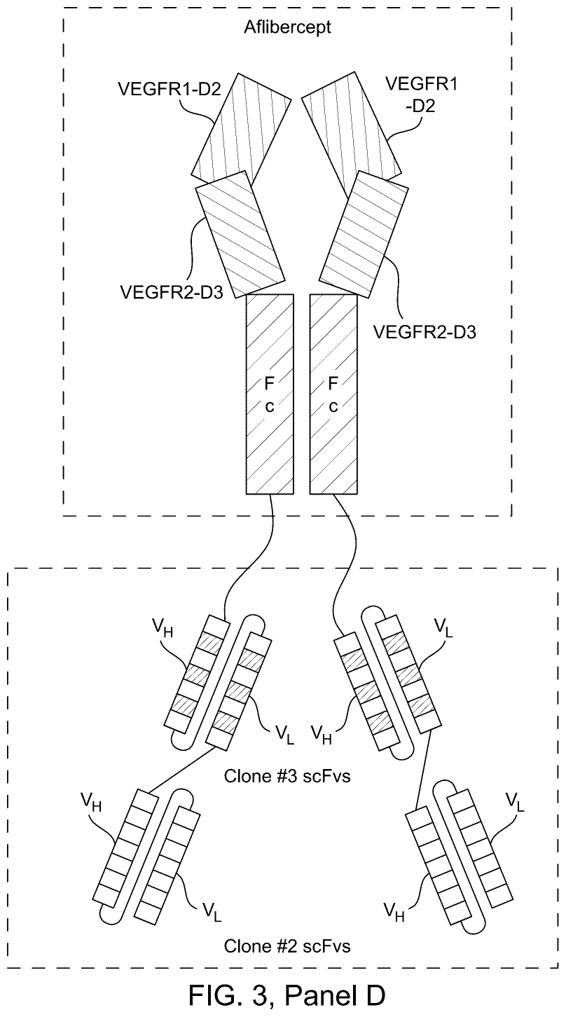

Suitable Tie2 binders to generate the bispecific antibodies of the invention are those disclosed in Damiano, J., et al., WO/2021/102173 filed Nov. 19, 2020 which are agonistic anti-Tie2 monoclonal antibodies. In one embodiment, the Tie2 binders used to generate the bispecific antibodies of the invention are the variable heavy and light chain domains of Clone #2 disclosed therein as SEQ ID NOs: 244 and 245 respectively. In another embodiment, the Tie2 binders used to generate the bispecific antibodies of the invention are the variable heavy and light chain domains of Clone #3 disclosed therein as SEQ ID Nos: 246 and 247, respectively. WO/2021/102173 and the sequences therein is hereby incorporated by reference in its entirety. See FIG. 1 and Table 1.

In some embodiments, suitable VEGF binders to generate the bispecific antibodies of the invention utilize the ligand-binding sequences from aflibercept or Eylea® (Regeneron-Bayer HealthCare, Tarrytown, NY, US) or from the antigen-binding sequences from ranibizumab or Lucentis® (Genentech Inc., a member of the Roche Group, CA, US). In other embodiments, the antigen-binding sequences from the B20 anti-VEGF monoclonal antibody was used (see Liang, W C, et al., JBC, 2006: 281, 951-961). See FIG. 1 and Table 1.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995).

The bispecific antibodies of the invention described herein, as well as any of the antibodies for use in a method described herein, may have any of the features, singly or in combination, described herein.

In one embodiment, KD is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER™ multi-well plates (Thermo Scientific) are coated overnight with 5 ug/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 μM [¹²⁵I]-antigen are mixed with serial dilutions of a Fab of interest (see Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20™) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, KD is measured using a BIACORE™ surface plasmon resonance (SPR) assay. For example, an assay using a BIACORE™-2000 or a BIACORE™-3000 (BIAcore, Inc., Piscataway, N.J.) is performed at 25° C. with immobilized antigen CM5 chips at about 10 response units (RU). In one embodiment, carboxymethylated dextran biosensor chips (CM5, BIAcore, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 ug/ml (about 0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN™-20) surfactant (PBST) at 25° C. at a flow rate of approximately 25 μl/min. Association rates (kₒₙ) and dissociation rates (k_{off}) are calculated using a simple one-to-one Langmuir binding model (BIACORE™ Evaluation Software version 3.2) by simultaneously fitting the association and dissociation sensorgrams. The equilibrium dissociation constant (KD) is calculated as the ratio k_{off}/kₒₙ. See, for example, Chen et al., J. Mol. Biol. 293:865-881 (1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette. KID may also be measured using a BIACORE™ SPR assay, known in the art.

The amino acid sequences of the bispecific antibodies of the invention are as disclosed in Table 1 herein and in the sequence listing:

TABLE 1

| Con- | | SEQ |
|---|---|---|
| struct | | ID |
| ID | AMINO ACID SEQUENCE | NO: |
| #1 | DVQLVESGGGVVRPGESLRLSCTASGFTFDSYGMSWVRQAPGKGLEWVSSINVGDNTYYADSVRGRFIISRDSSRNTLYL | 1 |
| Heavy | QMNSLTAEDTAVYYCANWNSFFDYWGLGTLVTVSSASTKGPSVFPLAPSSKSTSGGTAALGCLVKDYFPEPVTVSWNSGA | |
| chain | LTSGVHTFPAVLQSSGLYSLSSVVTVPSSSLGTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTKPREEQYNSTYRVVSVLTVLHQDWLNGKEYKCK | |
| | VSNKALGAPIEKTISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGKSDTGRPFVEMYSEIPEIIHMTEGRELVIPCRVTSP | |
| | NITVTLKKFPLDTLIPDGKRIIWDSRKGFIISNATYKEIGLLTCEATVNGHLYKTNYLTHRQTNTIIDVVLSPSHGIELS | |
| | VGEKLVLNCTARTELNVGIDFNWEYPSSKHQHKKLVNRDLKTQSGSEMKKFLSTLTIDGVTRSDQGLYTCAASSGLMTKK | |
| | NSTFVRVHEK | |
| #1 | EIVLTQSPGTLSLSPGEIATLSCRASQNVRSDLAWYQQKPGQAPRLLIYDASNRATGIPDRFSGSGSGTDFTLTISSLQS | 2 |
| Light | EDFAVYYCQQYSNWPPLTFGGGTKVEIKRTVAAPSVFIFPPSDEQLKSGTASVVCLLNNFYPREAKVQWKVDNALQSGNS | |
| Chain | QESVTEQDSKDSTYSLSSTLTLSKADYEKHKVYACEVTHQGLSSPVTKSFNRGEC | |
| (kappa) | | |
| #2 | DVQLVESGGGVVRPGESLRLSCTASGFTFDSYGMSWVRQAPGKGLEWVSSINVGDNTYYADSVRGRFIISRDSSRNTLYL | 3 |
| Heavy | QMNSLTAEDTAVYYCANWNSFFDYWGLGTLVTVSSASTKGPSVFPLAPSSKSTSGGTAALGCLVKDYFPEPVTVSWNSGA | |
| chain | LTSGVHTFPAVLQSSGLYSLSSVVTVPSSSLGTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTKPREEQYNSTYRVVSVLTVLHQDWLNGKEYKCK | |
| | VSNKALGAPIEKTISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGKGGGGSGGGGSDIQMTQSPSSLSASVGDRVTITCRA | |
| | SQVIRRSLAWYQQKPGKAPKLLIYAASNLASGVPSRFSGSGSGTDFTLTISSLQPEDFATYYCQQSNTSPLTFGQGTKVE | |
| | IKGQSSRSSGGGGSSGGGGSEVQLVESGGGLVQPGGSLRLSCAASGFSINGSWIFWVRQAPGKGLEWVGAIWPFGGYTHY | |
| | ADSVKGRFTISADTSKNTAYLQMNSLRAEDTAVYYCARWGHSTSPWAMDYWGQGTLVTVSS | |
| #2 | EIVLTQSPGTLSLSPGEIATLSCRASQNVRSDLAWYQQKPGQAPRLLIYDASNRATGIPDRFSGSGSGTDFTLTISSLQS | 2 |
| Light | EDFAVYYCQQYSNWPPLTFGGGTKVEIKRTVAAPSVFIFPPSDEQLKSGTASVVCLLNNFYPREAKVQWKVDNALQSGNS | |
| Chain | QESVTEQDSKDSTYSLSSTLTLSKADYEKHKVYACEVTHQGLSSPVTKSFNRGEC | |
| (kappa) | | |
| #3 | DVQLVESGGGVVRPGESLRLSCTASGFTFDSYGMSWVRQAPGKGLEWVSSINVGDNTYYADSVRGRFIISRDSSRNTLYL | 4 |
| Heavy | QMNSLTAEDTAVYYCANWNSFFDYWGLGTLVTVSSASTKGPSVFPLAPSSKSTSGGTAALGCLVKDYFPEPVTVSWNSGA | |
| chain | LTSGVHTFPAVLQSSGLYSLSSVVTVPSSSLGTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTKPREEQYNSTYRVVSVLTVLHQDWLNGKEYKCK | |
| | VSNKALGAPIEKTISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGKGGGGSGGGGSGGGGSSDTGRPFVEMYSEIPEIIHM | |
| | TEGRELVIPCRVTSPNITVTLKKFPLDTLIPDGKRIIWDSRKGFIISNATYKEIGLLTCEATVNGHLYKTNYLTHRQTNT | |
| | IIDVVLSPSHGIELSVGEKLVLNCTARTELNVGIDFNWEYPSSKHQHKKLVNRDLKTQSGSEMKKFLSTLTIDGVTRSDQ | |
| | GLYTCAASSGLMTKKNSTFVRVHEK | |
| #3 | EIVLTQSPGTLSLSPGEIATLSCRASQNVRSDLAWYQQKPGQAPRLLIYDASNRATGIPDRFSGSGSGTDFTLTISSLQS | 2 |
| Light | EDFAVYYCQQYSNWPPLTFGGGTKVEIKRTVAAPSVFIFPPSDEQLKSGTASVVCLLNNFYPREAKVQWKVDNALQSGNS | |
| chain | QESVTEQDSKDSTYSLSSTLTLSKADYEKHKVYACEVTHQGLSSPVTKSFNRGEC | |
| (kappa) | | |
| #4 | DVQLVESGGGVVRPGESLRLSCTASGFTFDSYGMSWVRQAPGKGLEWVSSINVGDNTYYADSVRGRFIISRDSSRNTLYL | 5 |
| Heavy | QMNSLTAEDTAVYYCANWNSFFDYWGLGTLVTVSSASTKGPSVFPLAPSSKSTSGGTAALGCLVKDYFPEPVTVSWNSGA | |
| chain | LTSGVHTFPAVLQSSGLYSLSSVVTVPSSSLGTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTKPREEQYNSTYRVVSVLTVLHQDWLNGKEYKCK | |
| | VSNKALGAPIEKTISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGKGGGGSGGGGSGGGGSGGGGSDIQLTQSPSSLSASV | |
| | GDRVTITCSASQDISNYLNWYQQKPGKAPKVLIYFTSSLHSGVPSRFSGSGSGTDFTLTISSLQPEDFATYYCQQYSTVP | |
| | WTFGQGTKVEIKRTVAAPSVFIFPPSDEQLKSGTASVVCLLNNFYPREAKVQWKVDNALQSGNSQESVTEQDSKDSTYSL | |
| | SSTLTLSKADYEKHKVYACEVTHQGLSSPVTKSFNRGECGGGGSGGGGSGGGGSGGGGSEVQLVESGGGLVQPGGSLRLS | |
| | CAASGYDFTHYGMNWVRQAPGKGLEWVGWINTYTGEPTYAADFKRRFTFSLDTSKSTAYLQMNSLRAEDTAVYYCAKYPY | |
| | YYGTSHWYFDVWGQGTLVTVSSASTKGPSVFPLAPSSKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSCDKTHL | |
| #4 | EIVLTQSPGTLSLSPGEIATLSCRASQNVRSDLAWYQQKPGQAPRLLIYDASNRATGIPDRFSGSGSGTDFTLTISSLQS | 2 |
| Light | EDFAVYYCQQYSNWPPLTFGGGTKVEIKRTVAAPSVFIFPPSDEQLKSGTASVVCLLNNFYPREAKVQWKVDNALQSGNS | |
| chain | QESVTEQDSKDSTYSLSSTLTLSKADYEKHKVYACEVTHQGLSSPVTKSFNRGEC | |
| (kappa) | | |
| #5 | DVQLVESGGGVVRPGESLRLSCTASGFTFDSYGMSWVRQAPGKGLEWVSSINVGDNTYYADSVRGRFIISRDSSRNTLYL | 6 |
| Heavy | QMNSLTAEDTAVYYCANWNSFFDYWGLGTLVTVSSASTKGPSVFPLAPSSKSTSGGTAALGCLVKDYFPEPVTVSWNSGA | |
| chain | LTSGVHTFPAVLQSSGLYSLSSVVTVPSSSLGTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTKPREEQYNSTYRVVSVLTVLHQDWLNGKEYKCK | |
| | VSNKALGAPIEKTISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGKGGGGSGGGGSGGGGSGGGGSEVQLVESGGGLVQPG | |
| | GSLRLSCAASGYDFTHYGMNWVRQAPGKGLEWVGWINTYTGEPTYAADFKRRFTFSLDTSKSTAYLQMNSLRAEDTAVYY | |
| | CAKYPYYYGTSHWYFDVWGQGTLVTVSSASTKGPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPKSCDKTHGGGGSGGGGSGGGGSGGGGSDIQLT | |
| | QSPSSLSASVGDRVTITCSASQDISNYLNWYQQKPGKAPKVLIYFTSSLHSGVPSRFSGSGSGTDFTLTISSLQPEDFAT | |
| | YYCQQYSTVPWTFGQGTKVEIKRTVAAPSVFIFPPSDEQLKSGTASVVCLLNNFYPREAKVQWKVDNALQSGNSQESVTE | |
| | QDSKDSTYSLSSTLTLSKADYEKHKVYACEVTHQGLSSPVTKSFNRGEC | |
| #5 | EIVLTQSPGTLSLSPGEIATLSCRASQNVRSDLAWYQQKPGQAPRLLIYDASNRATGIPDRFSGSGSGTDFTLTISSLQS | 2 |
| Light | EDFAVYYCQQYSNWPPLTFGGGTKVEIKRTVAAPSVFIFPPSDEQLKSGTASVVCLLNNFYPREAKVQWKVDNALQSGNS | |
| chain | QESVTEQDSKDSTYSLSSTLTLSKADYEKHKVYACEVTHQGLSSPVTKSFNRGEC | |
| (kappa) | | |
| #6 | SDTGRPFVEMYSEIPEIIHMTEGRELVIPCRVTSPNITVTLKKFPLDTLIPDGKRIIWDSRKGFIISNATYKEIGLLTCE | 7 |
| | ATVNGHLYKTNYLTHRQTNTIIDVVLSPSHGIELSVGEKLVLNCTARTELNVGIDFNWEYPSSKHQHKKLVNRDLKTQSG | |
| | SEMKKFLSTLTIDGVTRSDQGLYTCAASSGLMTKKNSTFVRVHEKDKTHTCPPCPAPEAAGGPSVFLFPPKPKDTLMISR | |
| | TPEVTCVVVDVSHEDPEVKFNWYVDGVEVHNAKTKPREEQYNSTYRVVSVLTVLHQDWLNGKEYKCKVSNKALGAPIEKT | |
| | ISKAKGQPREPQVYTLPPSRDELTKNQVSLTCLVKGFYPSDIAVEWESNGQPENNYKTTPPVLDSDGSFFLYSKLTVDKS | |
| | RWQQGNVFSCSVMHEALHNHYTQKSLSLSPGGGGGSGGGGSEIVLTQSPGTLSLSPGEIATLSCRASQNVRSDLAWYQQK | |
| | PGQAPRLLIYDASNRATGIPDRFSGSGSGTDFTLTISSLQSEDFAVYYCQQYSNWPPLTFGGGTKVEIKGQSSRSSGGGG | |
| | SSGGGGSDVQLVESGGGVVRPGESLRLSCTASGFTFDSYGMSWVRQAPGKGLEWVSSINVGDNTYYADSVRGRFIISRDS | |
| | SRNTLYLQMNSLTAEDTAVYYCANWNSFFDYWGLGTLVTVSS | |
| #7 | SDTGRPFVEMYSEIPEIIHMTEGRELVIPCRVTSPNITVTLKKFPLDTLIPDGKRIIWDSRKGFIISNATYKEIGLLTCE | 8 |
| | ATVNGHLYKTNYLTHRQTNTIIDVVLSPSHGIELSVGEKLVLNCTARTELNVGIDFNWEYPSSKHQHKKLVNRDLKTQSG | |
| | SEMKKFLSTLTIDGVTRSDQGLYTCAASSGLMTKKNSTFVRVHEKDKTHTCPPCPAPEAAGGPSVFLFPPKPKDTLMISR | |
| | TPEVTCVVVDVSHEDPEVKFNWYVDGVEVHNAKTKPREEQYNSTYRVVSVLTVLHQDWLNGKEYKCKVSNKALGAPIEKT | |
| | ISKAKGQPREPQVYTLPPSRDELTKNQVSLTCLVKGFYPSDIAVEWESNGQPENNYKTTPPVLDSDGSFFLYSKLTVDKS | |
| | RWQQGNVFSCSVMHEALHNHYTQKSLSLSPGGGGGSGGGGSGGGGSEIVLTQSPGTLSLSPGEIATLSCRASQNVRSDLA | |
| | WYQQKPGQAPRLLIYDASNRATGIPDRFSGSGSGTDFTLTISSLQSEDFAVYYCQQYSNWPPLTFGGGTKVEIKGQSSRS | |
| | SGGGGSSGGGGSDVQLVESGGGVVRPGESLRLSCTASGFTFDSYGMSWVRQAPGKGLEWVSSINVGDNTYYADSVRGRFI | |
| | ISRDSSRNTLYLQMNSLTAEDTAVYYCANWNSFFDYWGLGTLVTVSS | |
| #8 | SDTGRPFVEMYSEIPEIIHMTEGRELVIPCRVTSPNITVTLKKFPLDTLIPDGKRIIWDSRKGFIISNATYKEIGLLTCE | 9 |
| | ATVNGHLYKTNYLTHRQTNTIIDVVLSPSHGIELSVGEKLVLNCTARTELNVGIDFNWEYPSSKHQHKKLVNRDLKTQSG | |
| | SEMKKFLSTLTIDGVTRSDQGLYTCAASSGLMTKKNSTFVRVHEKDKTHTCPPCPAPEAAGGPSVFLFPPKPKDTLMISR | |
| | TPEVTCVVVDVSHEDPEVKFNWYVDGVEVHNAKTKPREEQYNSTYRVVSVLTVLHQDWLNGKEYKCKVSNKALGAPIEKT | |
| | ISKAKGQPREPQVYTLPPSRDELTKNQVSLTCLVKGFYPSDIAVEWESNGQPENNYKTTPPVLDSDGSFFLYSKLTVDKS | |
| | RWQQGNVFSCSVMHEALHNHYTQKSLSLSPGGGGGSGGGGSGGGGSEIVLTQSPGTLSLSPGEIATLSCRASQNVRSDLA | |
| | WYQQKPGQAPRLLIYDASNRATGIPDRFSGSGSGTDFTLTISSLQSEDFAVYYCQQYSNWPPLTFGGGTKVEIKGQSSRS | |
| | SGGGGSSGGGGSDVQLVESGGGVVRPGESLRLSCTASGFTFDSYGMSWVRQAPGKGLEWVSSINVGDNTYYADSVRGRFI | |
| | ISRDSSRNTLYLQMNSLTAEDTAVYYCANWNSFFDYWGLGTLVTVSSGGGGSGGGGSGGGGSEIVLTQSPGTVSLSPGER | |
| | VTLSCRASQPIDVYLAWYQQKPGQAPRLLIYGANRRAIDIPDRFSGSGSGTEFTLTINSLQNEDFAVYFCQQYMTWPPVT | |
| | FGGGTKVEIKGQSSRSSGGGGSSGGGGSDVQLVESGGGVVRPGESLRLSCAASGFSFINFAMTWVRQAPGEGPEWVSLIS | |
| | DDGRGNRPYYADSVKGRFTISRDNSKNILYLQMNSLRVEDTATYYCAKVSWDVFFDYWGLGTVVTVSS | |
| #9 | SDTGRPFVEMYSEIPEIIHMTEGRELVIPCRVTSPNITVTLKKFPLDTLIPDGKRIIWDSRKGFIISNATYKEIGLLTCE | 10 |
| | ATVNGHLYKTNYLTHRQTNTIIDVVLSPSHGIELSVGEKLVLNCTARTELNVGIDFNWEYPSSKHQHKKLVNRDLKTQSG | |
| | SEMKKFLSTLTIDGVTRSDQGLYTCAASSGLMTKKNSTFVRVHEKDKTHTCPPCPAPEAAGGPSVFLFPPKPKDTLMISR | |
| | TPEVTCVVVDVSHEDPEVKFNWYVDGVEVHNAKTKPREEQYNSTYRVVSVLTVLHQDWLNGKEYKCKVSNKALGAPIEKT | |
| | ISKAKGQPREPQVYTLPPSRDELTKNQVSLTCLVKGFYPSDIAVEWESNGQPENNYKTTPPVLDSDGSFFLYSKLTVDKS | |
| | RWQQGNVFSCSVMHEALHNHYTQKSLSLSPGGGGGSGGGGSGGGGSEIVLTQSPGTVSLSPGERVTLSCRASQPIDVYLA | |
| | WYQQKPGQAPRLLIYGANRRAIDIPDRFSGSGSGTEFTLTINSLQNEDFAVYFCQQYMTWPPVTFGGGTKVEIKGQSSRS | |
| | SGGGGSSGGGGSDVQLVESGGGVVRPGESLRLSCAASGFSFINFAMTWVRQAPGEGPEWVSLISDDGRGNRPYYADSVKG | |
| | RFTISRDNSKNILYLQMNSLRVEDTATYYCAKVSWDVFFDYWGLGTVVTVSSGGGGSGGGGSGGGGSEIVLTQSPGTLSL | |
| | SPGEIATLSCRASQNVRSDLAWYQQKPGQAPRLLIYDASNRATGIPDRFSGSGSGTDFTLTISSLQSEDFAVYYCQQYSN | |
| | WPPLTFGGGTKVEIKGQSSRSSGGGGSSGGGGSDVQLVESGGGVVRPGESLRLSCTASGFTFDSYGMSWVRQAPGKGLEW | |
| | VSSINVGDNTYYADSVRGRFIISRDSSRNTLYLQMNSLTAEDTAVYYCANWNSFFDYWGLGTLVTVSS | |
| #10 | DVQLVESGGGVVRPGESLRLSCTASGFTFDSYGMSWVRQAPGKGLEWVSSINVGDNTYYADSVRGRFIISRDSSRNTLYL | 11 |
| Heavy | QMNSLTAEDTAVYYCANWNSFFDYWGLGTLVTVSSASTKGPSVFPLAPSSKSTSGGTAALGCLVKDYFPEPVTVSWNSGA | |
| chain | LTSGVHTFPAVLQSSGLYSLSSVVTVPSSSLGTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTKPREEQYNSTYRVVSVLTVLHQDWLNGKEYKCK | |
| | VSNKALGAPIEKTISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGKGGGGSGGGGSDIQLTQSPSSLSASVGDRVTITCSA | |
| | SQDISNYLNWYQQKPGKAPKVLIYFTSSLHSGVPSRFSGSGSGTDFTLTISSLQPEDFATYYCQQYSTVPWTFGQGTKVE | |
| | IKRTVAAPSVFIFPPSDEQLKSGTASVVCLLNNFYPREAKVQWKVDNALQSGNSQESVTEQDSKDSTYSLSSTLTLSKAD | |
| | YEKHKVYACEVTHQGLSSPVTKSFNRGECGQSSRSSGGGGSSGGGGSEVQLVESGGGLVQPGGSLRLSCAASGYDFTHYG | |
| | MNWVRQAPGKGLEWVGWINTYTGEPTYAADFKRRFTFSLDTSKSTAYLQMNSLRAEDTAVYYCAKYPYYYGTSHWYFDVW | |
| | GQGTLVTVSSASTKGPSVFPLAPSSKSTSGGTAALGCLVKDYFPEPVTVSWNSGALTSGVHTFPAVLQSSGLYSLSSVVT | |
| | VPSSSLGTQTYICNVNHKPSNTKVDKKVEPKSCDKTHL | |
| #10 | EIVLTQSPGTLSLSPGEIATLSCRASQNVRSDLAWYQQKPGQAPRLLIYDASNRATGIPDRFSGSGSGTDFTLTISSLQS | 2 |
| Light | EDFAVYYCQQYSNWPPLTFGGGTKVEIKRTVAAPSVFIFPPSDEQLKSGTASVVCLLNNFYPREAKVQWKVDNALQSGNS | |
| chain | QESVTEQDSKDSTYSLSSTLTLSKADYEKHKVYACEVTHQGLSSPVTKSFNRGEC | |
| (kappa) | | |
| #11 | DVQLVESGGGVVRPGESLRLSCTASGFTFDSYGMSWVRQAPGKGLEWVSSINVGDNTYYADSVRGRFIISRDSSRNTLYL | 12 |
| Heavy | QMNSLTAEDTAVYYCANWNSFFDYWGLGTLVTVSSASTKGPSVFPLAPSSKSTSGGTAALGCLVKDYFPEPVTVSWNSGA | |
| chain | LTSGVHTFPAVLQSSGLYSLSSVVTVPSSSLGTQTYICNVNHKPSNTKVDKKVEPKSCDKTHTCPPCPAPEAAGGPSVFL | |
| | FPPKPKDTLMASRTPEVTCVVVDVSHEDPEVKFNWYVDGVEVHNAKTKPREEQYNSTYRVVSVLTVLAQDWLNGKEYKCK | |
| | VSNKALGAPIEKTISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNAYTQKSLSLSPGKGGGGSGGGGSDIQLTQSPSSLSASVGDRVTITCSA | |
| | SQDISNYLNWYQQKPGKAPKVLIYFTSSLHSGVPSRFSGSGSGTDFTLTISSLQPEDFATYYCQQYSTVPWTFGQGTKVE | |
| | IKGQSSRSSGGGGSSGGGGSEVQLVESGGGLVQPGGSLRLSCAASGYDFTHYGMNWVRQAPGKGLEWVGWINTYTGEPTY | |
| | AADFKRRFTFSLDTSKSTAYLQMNSLRAEDTAVYYCAKYPYYYGTSHWYFDVWGQGTLVTVSS | |
| #11 | EIVLTQSPGTLSLSPGEIATLSCRASQNVRSDLAWYQQKPGQAPRLLIYDASNRATGIPDRFSGSGSGTDFTLTISSLQS | 2 |
| Light | EDFAVYYCQQYSNWPPLTFGGGTKVEIKRTVAAPSVFIFPPSDEQLKSGTASVVCLLNNFYPREAKVQWKVDNALQSGNS | |
| chain | QESVTEQDSKDSTYSLSSTLTLSKADYEKHKVYACEVTHQGLSSPVTKSFNRGEC | |
| (kappa) | | |
| #12 | SDTGRPFVEMYSEIPEIIHMTEGRELVIPCRVTSPNITVTLKKFPLDTLIPDGKRIIWDSRKGFIISNATYKEIGLLTCE | 13 |
| | ATVNGHLYKTNYLTHRQTNTIIDVVLSPSHGIELSVGEKLVLNCTARTELNVGIDFNWEYPSSKHQHKKLVNRDLKTQSG | |
| | SEMKKFLSTLTIDGVTRSDQGLYTCAASSGLMTKKNSTFVRVHEKDKTHTCPPCPAPEAAGGPSVFLFPPKPKDTLMISR | |
| | TPEVTCVVVDVSHEDPEVKFNWYVDGVEVHNAKTKPREEQYNSTYRVVSVLTVLHQDWLNGKEYKCKVSNKALGAPIEKT | |
| | ISKAKGQPREPQVYTLPPSRDELTKNQVSLTCLVKGFYPSDIAVEWESNGQPENNYKTTPPVLDSDGSFFLYSKLTVDKS | |
| | RWQQGNVFSCSVMHEALHNHYTQKSLSLSPGGGGGSGGGGSGGGGSCSKPEVKKDVKTNSPKHNVNCIYTQTGLSSSPVT | |
| | VVSSLSYLGSSQLVAPFTHVGSTLAGSNWSVTVPEPFYDKVLCGLAATGGSTSKSSPALPFVSPGKTSASSVTSSVTVLT | |
| | GLGWYDFFSNWNACYYVATDEATLSNMQLYLTNRSSDRSIIFRGRVSDAYYTNDGVNISSVWELGKGPAQRVWSMGYSDF | |
| | TFGSATCSLRLSEGPRVVGGGSEVLQVDSGGGGSSGGGGSSRSSQGCEGRNFSKTVPSSLGQHTVECAYVKHKEYDAKSL | |
| | TLTSSLSYTSDKSDQETVSEQSNGSQLANDVKWQVKAERPYFNNLLCVVSATGSKLQEDSPPFIFVSPAAVTRKIEVKTG | |
| | GGFTLPPWNSYQQCYYVAFDESQLSSITLTFDTGSGSGSFRDPIGTARNSADYILLRPAQGPKQQYWALDSRVNQSARCS | |
| | LTAIEGPSLSLTGPSQTLVIE | |
| #13 | DVQLVESGGGVVRPGESLRLSCTASGFTFDSYGMSWVRQAPGKGLEWVSSINVGDNTYYADSVRGRFIISRDSSRNTLYL | 14 |
| Heavy | QMNSLTAEDTAVYYCANWNSFFDYWGLGTLVTVSSASTKGPSVFPLAPSSKSTSGGTAALGCLVKDYFPEPVTVSWNSGA | |
| chain | LTSGVHTFPAVLQSSGLYSLSSVVTVPSSSLGTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTKPREEQYNSTYRVVSVLTVLHQDWLNGKEYKCK | |
| | VSNKALGAPIEKTISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGKKEHVRVFTSNKKTMLGSSAACTYLGQDSRTVGDIT | |
| | LTSLFKKMESGSQTKLDRNVLKKHQHKSSPYEWNFDIGVNLETRATCNLVLKEGVSLEIGHSPSLVVDIITNTQRHTLYN | |
| | TKYLHGNVTAECTLLGIEKYTANSIIFGKRSDWIIRKGDPILTDLPFKKLTVTINPSTVRCPIVLERGETMHIIEPIESY | |
| | MEVFPRGTDS | |
| #13 | EIVLTQSPGTLSLSPGEIATLSCRASQNVRSDLAWYQQKPGQAPRLLIYDASNRATGIPDRFSGSGSGTDFTLTISSLQS | 2 |
| Light | EDFAVYYCQQYSNWPPLTFGGGTKVEIKRTVAAPSVFIFPPSDEQLKSGTASVVCLLNNFYPREAKVQWKVDNALQSGNS | |
| chain | QESVTEQDSKDSTYSLSSTLTLSKADYEKHKVYACEVTHQGLSSPVTKSFNRGEC | |
| (kappa) | | |
| #14 | DVQLVESGGGVVRPGESLRLSCTASGFTFDSYGMSWVRQAPGKGLEWVSSINVGDNTYYADSVRGRFIISRDSSRNTLYL | 15 |
| Heavy | QMNSLTAEDTAVYYCANWNSFFDYWGLGTLVTVSSASTKGPSVCPLAPSSKSTSGGTAALGCLVKDYFPEPVTVSWNSGA | |
| chain | LTSGVHTFPAVLQSSGLYSLSSVVTVPSSSLGTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTKPREEQYNSTYRVVSVLTVLHQDWLNGKEYKCK | |
| | VSNKALGAPIEKTISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGK | |
| #14 | EIVLTQSPGTLSLSPGEIATLSCRASQNVRSDLAWYQQKPGQAPRLLIYDASNRATGIPDRFSGSGSGTDFTLTISSLQS | 16 |
| Light | EDFAVYYCQQYSNWPPLTFGGGTKVEIKRTVAAPSVFIFPPCDEQLKSGTASVVCLLNNFYPREAKVQWKVDNALQSGNS | |
| chain | QESVTEQDSKDSTYSLSSTLTLSKADYEKHKVYACEVTHQGLSSPVTKSFNRGECGGGGSGGGGSDIQLTQSPSSLSASV | |
| (kappa) | GDRVTITCSASQDISNYLNWYQQKPGKAPKVLIYFTSSLHSGVPSRFSGSGSGTDFTLTISSLQPEDFATYYCQQYSTVP | |
| | WTFGQGTKVEIKGQSSRSSGGGGSSGGGGSEVQLVESGGGLVQPGGSLRLSCAASGYDFTHYGMNWVRQAPGKGLEWVGW | |
| | INTYTGEPTYAADFKRRFTFSLDTSKSTAYLQMNSLRAEDTAVYYCAKYPYYYGTSHWYFDVWGQGTLVTVSS | |
| #15 | DVQLVESGGGVVRPGESLRLSCTASGFTFDSYGMSWVRQAPGKGLEWVSSINVGDNTYYADSVRGRFIISRDSSRNTLYL | 17 |
| Heavy | QMNSLTAEDTAVYYCANWNSFFDYWGLGTLVTVSSASTKGPSVCPLAPSSKSTSGGTAALGCLVKDYFPEPVTVSWNSGA | |
| chain | LTSGVHTFPAVLQSSGLYSLSSVVTVPSSSLGTQTYICNVNHKPSNTKVDKKVEPKSCDKTHTCPPCPAPEAAGGPSVFL | |
| | FPPKPKDTLMASRTPEVTCVVVDVSHEDPEVKFNWYVDGVEVHNAKTKPREEQYNSTYRVVSVLTVLAQDWLNGKEYKCK | |
| | VSNKALGAPIEKTISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNAYTQKSLSLSPGKGGGGSGGGGSDIQLTQSPSSLSASVGDRVTITCSA | |
| | SQDISNYLNWYQQKPGKAPKVLIYFTSSLHSGVPSRFSGSGSGTDFTLTISSLQPEDFATYYCQQYSTVPWTFGQGTKVE | |
| | IKGQSSRSSGGGGSSGGGGSEVQLVESGGGLVQPGGSLRLSCAASGYDFTHYGMNWVRQAPGKGLEWVGWINTYTGEPTY | |
| | AADFKRRFTFSLDTSKSTAYLQMNSLRAEDTAVYYCAKYPYYYGTSHWYFDVWGQGTLVTVSS | |
| #15 | EIVLTQSPGTLSLSPGEIATLSCRASQNVRSDLAWYQQKPGQAPRLLIYDASNRATGIPDRFSGSGSGTDFTLTISSLQS | 18 |
| Light | EDFAVYYCQQYSNWPPLTFGGGTKVEIKRTVAAPSVFIFPPCDEQLKSGTASVVCLLNNFYPREAKVQWKVDNALQSGNS | |
| chain | QESVTEQDSKDSTYSLSSTLTLSKADYEKHKVYACEVTHQGLSSPVTKSFNRGEC | |
| (kappa) | | |
| #16 | DVQLVESGGGVVRPGESLRLSCTASGFTFDSYGMSWVRQAPGKGLEWVSSINVGDNTYYADSVRGRFIISRDSSRNTLYL | 19 |
| Heavy | QMNSLTAEDTAVYYCANWNSFFDYWGLGTLVTVSSASTKGPSVFPLAPSSKSTSGGTAALGCLVKDYFPEPVTVSWNSGA | |
| chain | LTSGVHTFPAVLQSSGLYSLSSVVTVPSSSLGTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTKPREEQYNSTYRVVSVLTVLHQDWLNGKEYKCK | |
| | VSNKALGAPIEKTISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGKGGGGSGGGGGGGGSGGGGSDIQLTQSPSSLSASVG | |
| | DRVTITCSASQDISNYLNWYQQKPGKAPKVLIYFTSSLHSGVPSRFSGSGSGTDFTLTISSLQPEDFATYYCQQYSTVPW | |
| | TFGQGTKVEIKRTVAAPSVFIFPPSDEQLKSGTASVVCLLNNFYPREAKVQWKVDNALQSGNSQESVTEQDSKDSTYSLS | |
| | STLTLSKADYEKHKVYACEVTHQGLSSPVTKSFNRGECGGGGSGGGGSGGGGSGGGGSGGGGSEVQLVESGGGLVQPGGS | |
| | LRLSCAASGYDFTHYGMNWVRQAPGKGLEWVGWINTYTGEPTYAADFKRRFTFSLDTSKSTAYLQMNSLRAEDTAVYYCA | |
| | KYPYYYGTSHWYFDVWGQGTLVTVSSASTKGPSVFPLAPSSKSTSGGTAALGCLVKDYFPEPVTVSWNSGALTSGVHTFP | |
| | AVLQSSGLYSLSSVVTVPSSSLGTQTYICNVNHKPSNTKVDKKVEPKSCDKTHL | |
| #16 | EIVLTQSPGTLSLSPGEIATLSCRASQNVRSDLAWYQQKPGQAPRLLIYDASNRATGIPDRFSGSGSGTDFTLTISSLQS | 2 |
| Light | EDFAVYYCQQYSNWPPLTFGGGTKVEIKRTVAAPSVFIFPPSDEQLKSGTASVVCLLNNFYPREAKVQWKVDNALQSGNS | |
| chain | QESVTEQDSKDSTYSLSSTLTLSKADYEKHKVYACEVTHQGLSSPVTKSFNRGEC | |
| (kappa) | | |
| #17 | DVQLVESGGGVVRPGESLRLSCTASGFTFDSYGMSWVRQAPGKGLEWVSSINVGDNTYYADSVRGRFIISRDSSRNTLYL | 20 |
| Heavy | QMNSLTAEDTAVYYCANWNSFFDYWGLGTLVTVSSASTKGPSVFPLAPSSKSTSGGTAALGCLVKDYFPEPVTVSWNSGA | |
| chain | LTSGVHTFPAVLQSSGLYSLSSVVTVPSSSLGTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTKPREEQYNSTYRVVSVLTVLHQDWLNGKEYKCK | |
| | VSNKALGAPIEKTISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGKGGGGSGGGGSGGGGSGGGGSDIQLTQSPSSLSASV | |
| | GDRVTITCSASQDISNYLNWYQQKPGKAPKVLIYFTSSLHSGVPSRFSGSGSGTDFTLTISSLQPEDFATYYCQQYSTVP | |
| | WTFGQGTKVEIKRTVAAPSVFIFPPSDEQLKSGTASVVCLLNNFYPREAKVQWKVDNALQSGNSQESVTEQDSKDSTYSL | |
| | SSTLTLSKADYEKHKVYACEVTHQGLSSPVTKSFNRGECGGGGSGGGGSGGGGSGGGGSGGGGSGGGGSEVQLVESGGGL | |
| | VQPGGSLRLSCAASGYDFTHYGMNWVRQAPGKGLEWVGWINTYTGEPTYAADFKRRFTFSLDTSKSTAYLQMNSLRAEDT | |
| | AVYYCAKYPYYYGTSHWYFDVWGQGTLVTVSSASTKGPSVFPLAPSSKSTSGGTAALGCLVKDYFPEPVTVSWNSGALTS | |
| | GVHTFPAVLQSSGLYSLSSVVTVPSSSLGTQTYICNVNHKPSNTKVDKKVEPKSCDKTHL | |
| #17 | EIVLTQSPGTLSLSPGEIATLSCRASQNVRSDLAWYQQKPGQAPRLLIYDASNRATGIPDRFSGSGSGTDFTLTISSLQS | 2 |
| Light | EDFAVYYCQQYSNWPPLTFGGGTKVEIKRTVAAPSVFIFPPSDEQLKSGTASVVCLLNNFYPREAKVQWKVDNALQSGNS | |
| chain | QESVTEQDSKDSTYSLSSTLTLSKADYEKHKVYACEVTHQGLSSPVTKSFNRGEC | |
| (kappa) | | |
| #18 | DVQLVESGGGVVRPGESLRLSCTASGFTFDSYGMSWVRQAPGKGLEWVSSINVGDNTYYADSVRGRFIISRDSSRNTLYL | 21 |
| Heavy | QMNSLTAEDTAVYYCANWNSFFDYWGLGTLVTVSSASTKGPSVFPLAPSSKSTSGGTAALGCLVKDYFPEPVTVSWNSGA | |
| chain | LTSGVHTFPAVLQSSGLYSLSSVVTVPSSSLGTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTKPREEQYNSTYRVVSVLTVLHQDWLNGKEYKCK | |
| | VSNKALGAPIEKTISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGKGGGGSGGGGSGGGGSGGGGSDIQLTQSPSSLSASV | |
| | GDRVTITCSASQDISNYLNWYQQKPGKAPKVLIYFTSSLHSGVPSRFSGSGSGTDFTLTISSLQPEDFATYYCQQYSTVP | |
| | WTFGQGTKVEIKRTVAAPSVFIFPPSDEQLKSGTASVVCLLNNFYPREAKVQWKVDNALQSGNSQESVTEQDSKDSTYSL | |
| | SSTLTLSKADYEKHKVYACEVTHQGLSSPVTKSFNRGECGGGGSGGGGSGGGGSGGGGSGGGGSGGGGSGGGGSEVQLVE | |
| | SGGGLVQPGGSLRLSCAASGYDFTHYGMNWVRQAPGKGLEWVGWINTYTGEPTYAADFKRRFTFSLDTSKSTAYLQMNSL | |
| | RAEDTAVYYCAKYPYYYGTSHWYFDVWGQGTLVTVSSASTKGPSVFPLAPSSKSTSGGTAALGCLVKDYFPEPVTVSWNS | |
| | GALTSGVHTFPAVLQSSGLYSLSSVVTVPSSSLGTQTYICNVNHKPSNTKVDKKVEPKSCDKTHL | |
| #18 | EIVLTQSPGTLSLSPGEIATLSCRASQNVRSDLAWYQQKPGQAPRLLIYDASNRATGIPDRFSGSGSGTDFTLTISSLQS | 2 |
| Light | EDFAVYYCQQYSNWPPLTFGGGTKVEIKRTVAAPSVFIFPPSDEQLKSGTASVVCLLNNFYPREAKVQWKVDNALQSGNS | |
| chain | QESVTEQDSKDSTYSLSSTLTLSKADYEKHKVYACEVTHQGLSSPVTKSFNRGEC | |
| (kappa) | | |
| #19 | DVQLVESGGGVVRPGESLRLSCTASGFTFDSYGMSWVRQAPGKGLEWVSSINVGDNTYYADSVRGRFIISRDSSRNTLYL | 22 |
| Heavy | QMNSLTAEDTAVYYCANWNSFFDYWGLGTLVTVSSASTKGPSVFPLAPSSKSTSGGTAALGCLVKDYFPEPVTVSWNSGA | |
| chain | LTSGVHTFPAVLQSSGLYSLSSVVTVPSSSLGTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTKPREEQYNSTYRVVSVLTVLHQDWLNGKEYKCK | |
| | VSNKALGAPIEKTISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGKGGGGSGGGGSGGGGSGGGGSDIQLTQSPSSLSASV | |
| | GDRVTITCSASQDISNYLNWYQQKPGKAPKVLIYFTSSLHSGVPSRFSGSGSGTDFTLTISSLQPEDFATYYCQQYSTVP | |
| | WTFGQGTKVEIKRTVAAPSVFIFPPSDEQLKSGTASVVCLLNNFYPREAKVQWKVDNALQSGNSQESVTEQDSKDSTYSL | |
| | SSTLTLSKADYEKHKVYACEVTHQGLSSPVTKSFNRGECGGGGSGGGGGGGGSGGGGSGGGGSGGGGSGGGGSGGGGSGG | |
| | GGSGGGGSEVQLVESGGGLVQPGGSLRLSCAASGYDFTHYGMNWVRQAPGKGLEWVGWINTYTGEPTYAADFKRRFTFSL | |
| | DTSKSTAYLQMNSLRAEDTAVYYCAKYPYYYGTSHWYFDVWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQTYICNVNHKPSNTKVDKKVEPKSCDKTHL | |
| #19 | EIVLTQSPGTLSLSPGEIATLSCRASQNVRSDLAWYQQKPGQAPRLLIYDASNRATGIPDRFSGSGSGTDFTLTISSLQS | 2 |
| Light | EDFAVYYCQQYSNWPPLTFGGGTKVEIKRTVAAPSVFIFPPSDEQLKSGTASVVCLLNNFYPREAKVQWKVDNALQSGNS | |
| chain | QESVTEQDSKDSTYSLSSTLTLSKADYEKHKVYACEVTHQGLSSPVTKSFNRGEC | |
| (kappa) | | |
| #20 | DVQLVESGGGVVRPGESLRLSCTASGFTFDSYGMSWVRQAPGKGLEWVSSINVGDNTYYADSVRGRFIISRDSSRNTLYL | 23 |
| Heavy | QMNSLTAEDTAVYYCANWNSFFDYWGLGTLVTVSSASTKGPSVFPLAPSSKSTSGGTAALGCLVKDYFPEPVTVSWNSGA | |
| chain | LTSGVHTFPAVLQSSGLYSLSSVVTVPSSSLGTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTKPREEQYNSTYRVVSVLTVLHQDWLNGKEYKCK | |
| | VSNKALGAPIEKTISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGKGGGGSGGGGSGGGGSGGGGSGGGGSDIQLTQSPSS | |
| | LSASVGDRVTITCSASQDISNYLNWYQQKPGKAPKVLIYFTSSLHSGVPSRFSGSGSGTDFTLTISSLQPEDFATYYCQQ | |
| | YSTVPWTFGQGTKVEIKRTVAAPSVFIFPPSDEQLKSGTASVVCLLNNFYPREAKVQWKVDNALQSGNSQESVTEQDSKD | |
| | STYSLSSTLTLSKADYEKHKVYACEVTHQGLSSPVTKSFNRGECGGGGSGGGGSGGGGSGGGGSGGGGSGGGGSGGGGSG | |
| | GGGSGGGGSGGGGSEVQLVESGGGLVQPGGSLRLSCAASGYDFTHYGMNWVRQAPGKGLEWVGWINTYTGEPTYAADFKR | |
| | RFTFSLDTSKSTAYLQMNSLRAEDTAVYYCAKYPYYYGTSHWYFDVWGQGTLVTVSSASTKGPSVFPLAPSSKSTSGGTA | |
| | ALGCLVKDYFPEPVTVSWNSGALTSGVHTFPAVLQSSGLYSLSSVVTVPSSSLGTQTYICNVNHKPSNTKVDKKVEPKSC | |
| | DKTHL | |
| #20 | EIVLTQSPGTLSLSPGEIATLSCRASQNVRSDLAWYQQKPGQAPRLLIYDASNRATGIPDRFSGSGSGTDFTLTISSLQS | 2 |
| Light | EDFAVYYCQQYSNWPPLTFGGGTKVEIKRTVAAPSVFIFPPSDEQLKSGTASVVCLLNNFYPREAKVQWKVDNALQSGNS | |
| chain | QESVTEQDSKDSTYSLSSTLTLSKADYEKHKVYACEVTHQGLSSPVTKSFNRGEC | |
| (kappa) | | |
| #21 | DVQLVESGGGVVRPGESLRLSCTASGFTFDSYGMSWVRQAPGKGLEWVSSINVGDNTYYADSVRGRFIISRDSSRNTLYL | 24 |
| Heavy | QMNSLTAEDTAVYYCANWNSFFDYWGLGTLVTVSSASTKGPSVFPLAPSSKSTSGGTAALGCLVKDYFPEPVTVSWNSGA | |
| chain | LTSGVHTFPAVLQSSGLYSLSSVVTVPSSSLGTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTKPREEQYNSTYRVVSVLTVLHQDWLNGKEYKCK | |
| | VSNKALGAPIEKTISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGKGGGGSGGGGSGGGGSGGGGSEVQLVESGGGLVQPG | |
| | GSLRLSCAASGYDFTHYGMNWVRQAPGKGLEWVGWINTYTGEPTYAADFKRRFTFSLDTSKSTAYLQMNSLRAEDTAVYY | |
| | CAKYPYYYGTSHWYFDVWGQGTLVTVSSASTKGPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPKSCDKTHGGGGSGGGGSGGGGSGGGGSGGGGS | |
| | DIQLTQSPSSLSASVGDRVTITCSASQDISNYLNWYQQKPGKAPKVLIYFTSSLHSGVPSRFSGSGSGTDFTLTISSLQP | |
| | EDFATYYCQQYSTVPWTFGQGTKVEIKRTVAAPSVFIFPPSDEQLKSGTASVVCLLNNFYPREAKVQWKVDNALQSGNSQ | |
| | ESVTEQDSKDSTYSLSSTLTLSKADYEKHKVYACEVTHQGLSSPVTKSFNRGEC | |
| #21 | EIVLTQSPGTLSLSPGEIATLSCRASQNVRSDLAWYQQKPGQAPRLLIYDASNRATGIPDRFSGSGSGTDFTLTISSLQS | 2 |
| Light | EDFAVYYCQQYSNWPPLTFGGGTKVEIKRTVAAPSVFIFPPSDEQLKSGTASVVCLLNNFYPREAKVQWKVDNALQSGNS | |
| chain | QESVTEQDSKDSTYSLSSTLTLSKADYEKHKVYACEVTHQGLSSPVTKSFNRGEC | |
| (kappa) | | |
| #22 | DVQLVESGGGVVRPGESLRLSCTASGFTFDSYGMSWVRQAPGKGLEWVSSINVGDNTYYADSVRGRFIISRDSSRNTLYL | 25 |
| Heavy | QMNSLTAEDTAVYYCANWNSFFDYWGLGTLVTVSSASTKGPSVFPLAPSSKSTSGGTAALGCLVKDYFPEPVTVSWNSGA | |
| chain | LTSGVHTFPAVLQSSGLYSLSSVVTVPSSSLGTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTKPREEQYNSTYRVVSVLTVLHQDWLNGKEYKCK | |
| | VSNKALGAPIEKTISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGKGGGGSGGGGSGGGGSGGGGSEVQLVESGGGLVQPG | |
| | GSLRLSCAASGYDFTHYGMNWVRQAPGKGLEWVGWINTYTGEPTYAADFKRRFTFSLDTSKSTAYLQMNSLRAEDTAVYY | |
| | CAKYPYYYGTSHWYFDVWGQGTLVTVSSASTKGPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPKSCDKTHGGGGSGGGGSGGGGSGGGGSGGGGS | |
| | GGGGSDIQLTQSPSSLSASVGDRVTITCSASQDISNYLNWYQQKPGKAPKVLIYFTSSLHSGVPSRFSGSGSGTDFTLTI | |
| | SSLQPEDFATYYCQQYSTVPWTFGQGTKVEIKRTVAAPSVFIFPPSDEQLKSGTASVVCLLNNFYPREAKVQWKVDNALQ | |
| | SGNSQESVTEQDSKDSTYSLSSTLTLSKADYEKHKVYACEVTHQGLSSPVTKSFNRGEC | |
| #22 | EIVLTQSPGTLSLSPGEIATLSCRASQNVRSDLAWYQQKPGQAPRLLIYDASNRATGIPDRFSGSGSGTDFTLTISSLQS | 2 |
| Light | EDFAVYYCQQYSNWPPLTFGGGTKVEIKRTVAAPSVFIFPPSDEQLKSGTASVVCLLNNFYPREAKVQWKVDNALQSGNS | |
| chain | QESVTEQDSKDSTYSLSSTLTLSKADYEKHKVYACEVTHQGLSSPVTKSFNRGEC | |
| (kappa) | | |
| #23 | DVQLVESGGGVVRPGESLRLSCTASGFTFDSYGMSWVRQAPGKGLEWVSSINVGDNTYYADSVRGRFIISRDSSRNTLYL | 26 |
| Heavy | QMNSLTAEDTAVYYCANWNSFFDYWGLGTLVTVSSASTKGPSVFPLAPSSKSTSGGTAALGCLVKDYFPEPVTVSWNSGA | |
| chain | LTSGVHTFPAVLQSSGLYSLSSVVTVPSSSLGTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTKPREEQYNSTYRVVSVLTVLHQDWLNGKEYKCK | |
| | VSNKALGAPIEKTISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGKGGGGSGGGGSGGGGSGGGGSEVQLVESGGGLVQPG | |
| | GSLRLSCAASGYDFTHYGMNWVRQAPGKGLEWVGWINTYTGEPTYAADFKRRFTFSLDTSKSTAYLQMNSLRAEDTAVYY | |
| | CAKYPYYYGTSHWYFDVWGQGTLVTVSSASTKGPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPKSCDKTHGGGGSGGGGSGGGGSGGGGSGGGGS | |
| | GGGGSGGGGSDIQLTQSPSSLSASVGDRVTITCSASQDISNYLNWYQQKPGKAPKVLIYFTSSLHSGVPSRFSGSGSGTD | |
| | FTLTISSLQPEDFATYYCQQYSTVPWTFGQGTKVEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQGLSSPVTKSFNRGEC | |
| #23 | EIVLTQSPGTLSLSPGEIATLSCRASQNVRSDLAWYQQKPGQAPRLLIYDASNRATGIPDRFSGSGSGTDFTLTISSLQS | 2 |
| Light | EDFAVYYCQQYSNWPPLTFGGGTKVEIKRTVAAPSVFIFPPSDEQLKSGTASVVCLLNNFYPREAKVQWKVDNALQSGNS | |
| chain | QESVTEQDSKDSTYSLSSTLTLSKADYEKHKVYACEVTHQGLSSPVTKSFNRGEC | |
| (kappa) | | |
| #24 | DVQLVESGGGVVRPGESLRLSCTASGFTFDSYGMSWVRQAPGKGLEWVSSINVGDNTYYADSVRGRFIISRDSSRNTLYL | 27 |
| Heavy | QMNSLTAEDTAVYYCANWNSFFDYWGLGTLVTVSSASTKGPSVFPLAPSSKSTSGGTAALGCLVKDYFPEPVTVSWNSGA | |
| chain | LTSGVHTFPAVLQSSGLYSLSSVVTVPSSSLGTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTKPREEQYNSTYRVVSVLTVLHQDWLNGKEYKCK | |
| | VSNKALGAPIEKTISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGKGGGGSGGGGSGGGGSGGGGSEVQLVESGGGLVQPG | |
| | GSLRLSCAASGYDFTHYGMNWVRQAPGKGLEWVGWINTYTGEPTYAADFKRRFTFSLDTSKSTAYLQMNSLRAEDTAVYY | |
| | CAKYPYYYGTSHWYFDVWGQGTLVTVSSASTKGPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPKSCDKTHGGGGSGGGGSGGGGSGGGGSGGGGS | |
| | GGGGSGGGGSGGGGSGGGGSGGGGSDIQLTQSPSSLSASVGDRVTITCSASQDISNYLNWYQQKPGKAPKVLIYFTSSLH | |
| | SGVPSRFSGSGSGTDFTLTISSLQPEDFATYYCQQYSTVPWTFGQGTKVEIKRTVAAPSVFIFPPSDEQLKSGTASVVCL | |
| | LNNFYPREAKVQWKVDNALQSGNSQESVTEQDSKDSTYSLSSTLTLSKADYEKHKVYACEVTHQGLSSPVTKSFNRGEC | |
| #24 | EIVLTQSPGTLSLSPGEIATLSCRASQNVRSDLAWYQQKPGQAPRLLIYDASNRATGIPDRFSGSGSGTDFTLTISSLQS | 2 |
| Light | EDFAVYYCQQYSNWPPLTFGGGTKVEIKRTVAAPSVFIFPPSDEQLKSGTASVVCLLNNFYPREAKVQWKVDNALQSGNS | |
| chain | QESVTEQDSKDSTYSLSSTLTLSKADYEKHKVYACEVTHQGLSSPVTKSFNRGEC | |
| (kappa) | | |
| #25 | DVQLVESGGGVVRPGESLRLSCTASGFTFDSYGMSWVRQAPGKGLEWVSSINVGDNTYYADSVRGRFIISRDSSRNTLYL | 28 |
| Heavy | QMNSLTAEDTAVYYCANWNSFFDYWGLGTLVTVSSASTKGPSVFPLAPSSKSTSGGTAALGCLVKDYFPEPVTVSWNSGA | |
| chain | LTSGVHTFPAVLQSSGLYSLSSVVTVPSSSLGTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTKPREEQYNSTYRVVSVLTVLHQDWLNGKEYKCK | |
| | VSNKALGAPIEKTISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGKGGGGSGGGGSGGGGSGGGGSGGGGSEVQLVESGGG | |
| | LVQPGGSLRLSCAASGYDFTHYGMNWVRQAPGKGLEWVGWINTYTGEPTYAADFKRRFTFSLDTSKSTAYLQMNSLRAED | |
| | TAVYYCAKYPYYYGTSHWYFDVWGQGTLVTVSSASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKVDKKVEPKSCDKTHGGGGSGGGGGGGGSGGGGSG | |
| | GGGSGGGGSGGGGSGGGGGGGGSGGGGSDIQLTQSPSSLSASVGDRVTITCSASQDISNYLNWYQQKPGKAPKVLIYFTS | |
| | SLHSGVPSRFSGSGSGTDFTLTISSLQPEDFATYYCQQYSTVPWTFGQGTKVEIKRTVAAPSVFIFPPSDEQLKSGTASV | |
| | VCLLNNFYPREAKVQWKVDNALQSGNSQESVTEQDSKDSTYSLSSTLTLSKADYEKHKVYACEVTHQGLSSPVTKSFNRG | |
| | EC | |
| #25 | EIVLTQSPGTLSLSPGEIATLSCRASQNVRSDLAWYQQKPGQAPRLLIYDASNRATGIPDRFSGSGSGTDFTLTISSLQS | 2 |
| Light | EDFAVYYCQQYSNWPPLTFGGGTKVEIKRTVAAPSVFIFPPSDEQLKSGTASVVCLLNNFYPREAKVQWKVDNALQSGNS | |
| chain | QESVTEQDSKDSTYSLSSTLTLSKADYEKHKVYACEVTHQGLSSPVTKSFNRGEC | |
| (kappa) | | |

In certain embodiments, a bispecific antibody provided herein is a human antibody. Human antibodies can be produced using various techniques known in the art. Human antibodies are described generally in van Dijk et al., Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. For review of methods for obtaining human antibodies from transgenic animals, see Lonberg, Nat. Biotech. 23:1117-1125 (2005). See also, for example, U.S. Pat. Nos. 6,075,181 and 6,150,584 describing XENOMOUSE™ technology; U.S. Pat. No. 5,770,429 describing HUANTIBODIES™ technology; U.S. Pat. No. 7,041,870 describing K-M MOUSE™ technology, and U.S. Patent Application Publication No. US 2007/0061900, describing VELOCIMOUSE™ technology, and U.S. Pat. Nos. 9,809,642 and 9,380,769, describing OmniChicken™ technology). Human variable regions from intact antibodies generated by such animals may be further modified, for example, by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Bispecific antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al., in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naïve repertoire can be cloned (e.g., from human) to provide a single source of antibodies to a wide range of non-self and also self antigens without any immunization as described by Griffiths et al., EMBO J, 12: 725-734 (1993). Finally, naïve libraries can also be made synthetically by cloning unrearranged V-gene segments from stem cells and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

Techniques for making the bispecific antibodies of the invention include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. immunol., 152:5368 (1994)); and preparing trispecific antibodies as described, for example, in Tutt et al., J. Immunol. 147:60 (1991).

The antibody or fragment herein also includes a “Dual Acting FAb” or “DAF” comprising an antigen binding site that binds to Tie2 as well as VEGF (see, e.g., US 2008/0069820).

In certain embodiments, amino acid sequence variants (e.g., antibody variants including one or more amino acid residue alterations) of the bispecific antibodies provided herein are contemplated. For example, it may be desirable to improve the binding affinity and/or other biological properties of the bispecific antibody. Amino acid sequence variants of a bispecific antibody may be prepared by introducing appropriate modifications into the nucleotide sequence encoding the bispecific antibody, or by peptide synthesis. Such modifications include, for example, deletions from, and/or insertions into and/or substitutions of residues within the amino acid sequences of the bispecific antibody. Any combination of deletion, insertion, and substitution can be made to arrive at the final construct, provided that the final construct possesses the desired characteristics, for example, antigen binding to either Tie2 or VEGF.

In certain embodiments, bispecific antibody variants having one or more amino acid substitutions are provided. Sites of interest for substitutional mutagenesis include the CDRs and FRs. Conservative substitutions are contemplated, and such are well-known in the art.

Other amino acid substitutions are described below in reference to amino acid side chain classes. Amino acid substitutions may be introduced into a bispecific antibody of interest and the products screened for a desired activity, for example, retained/improved antigen binding, decreased immunogenicity, or improved or decreased ADCC or CDC. Amino acids may be grouped according to common side-chain properties:
(1) hydrophobic: Norleucine, Met, Ala, Val, Leu, lie:
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues and/or FR residues of a parent antibody (e.g., a humanized or human antibody). Generally, the resulting variant(s) selected for further study will have modifications (e.g., improvements) in certain biological properties (e.g., increased affinity, increased stability, increased expression, altered pI, and/or reduced immunogenicity) relative to the parent antibody and/or will have substantially retained certain biological properties of the parent antibody. An exemplary substitutional variant is an affinity matured antibody, which may be conveniently generated, for example, using phage display-based affinity maturation techniques such as those described herein. Briefly, one or more CDR residues are mutated and the variant antibodies displayed on phage and screened for a particular biological activity (e.g., binding affinity).

Alterations (e.g., substitutions) may be made in CDRs, for example, to improve antibody affinity. Such alterations may be made in CDR “hotspots,” i.e., residues encoded by codons that undergo mutation at high frequency during the somatic maturation process (see, e.g., Chowdhury, Methods Mol Biol. 207:179-196 (2008)), and/or residues that contact antigen, with the resulting variant VH or VL being tested for binding affinity. Affinity maturation by constructing and reselecting from secondary libraries has been described, for example, in Hoogenboom et al., in Methods in Molecular Biology 178:1-37 (O'Brien et al., ed., Human Press, Totowa, N.J., (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant VH and VL sequences provided above, each CDR either is unaltered, or contains no more than one, two or three amino acid substitutions.

In certain embodiments, substitutions, insertions, or deletions may occur within one or more FRs so long as such alterations do not substantially reduce the ability of the antibody to bind antigen. Such alterations may, for example, improve antibody affinity and/or stability (e.g., as assessed by an increased melting temperature).

In certain embodiments, one or more amino acid modifications may be introduced into the Fc region of a bispecific antibody provided herein, thereby generating an Fc region variant. The Fc region variant may comprise a human Fc region sequence (e.g., a human IgG1, IgG2, IgG3 or IgG4 Fc region) comprising an amino acid residue alteration (e.g., a substitution) at one or more amino acid positions. In certain embodiments, the invention contemplates a variant that possesses some but not all effector functions, which make it a desirable candidate for applications in which the half-life of the bispecific antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the bispecific antibody lacks FcγR binding (hence likely lacking ADCC activity) but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991).

Non-limiting examples of in vitro assays to assess ADCC activity of a bispecific antibody of interest are described in U.S. Pat. No. 5,500,362 (see, e.g., Hellstrom et al., Proc. Nat'l Acad. Sci. USA 83:7059-7063 (1986) and Hellstrom et al., Proc. Nat'l Acad. Sci. USA 82:1499-1502 (1985); U.S. Pat. No. 5,821,337; and Bruggemann et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc., Mountain View, Calif.; and CYTOTOX 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the bispecific antibody of interest may be assessed in vivo, for example, in an animal model such as that disclosed in Clynes et al., Proc. Nat'l Acad. Sci. USA 95:652-656 (1998). C1q binding assays may also be carried out to confirm that the bispecific antibody is unable to bind C1q and hence lacks CDC activity. See, for example, C1q and C3c binding ELISA in WO 2006/029879 and WO 2005/100402. To assess complement activation, a CDC assay may be performed (see, e.g., Gazzano-Santoro et al., J. Immunol Methods 202:163 (1996); Cragg et al., Blood 101:1045-1052 (2003); and Cragg et al., Blood 103:2738-2743 (2004)). FcRn binding and in vivo clearance/half life determinations can also be performed using methods known in the art (see, e.g., Petkova et al., Int'l. Immunol. 18(12):1759-1769 (2006)).

Bispecific antibodies of the invention may be engineered with reduced effector functions, such as decreased complement-dependent cytotoxicity (CDC), antibody-dependent cellular cytotoxicity (ADCC) and antibody-dependent cellular phagocytosis (ADCP) through a reduced affinity to the human FcγRIIIA and/or FcγRIIA and/or FcγRI and/or C1q binding. In some instances, such reduced effector functions are achieved by amino acid substitutions of one or more of the following Fc region residues: N297, L234, L235, D265 and P329, according to EU numbering as in Kabat. See U.S. Pat. Nos. 6,737,056, 7,332,581 and WO/2012/130831. In some embodiments, the substitution mutation is one or more of N297G, N297A, L234A, L235A, D265A, and/or P329G. In some embodiments, the substitution mutation is an N297A or an N297G substitution mutation. In some embodiments, the substitution mutation includes the so-called “DANA” Fc mutant with substitution of residues D265 and N297 to alanine (U.S. Pat. No. 7,332,581). In some embodiments, the substitution mutation includes the “LALA” Fc mutant with substitution of residues L234 and L235 to alanine (see Lund, J., et al., (1992) Mol. Immunol., 29, 53-59; and Tamm, A. and Schmidt, R. E. (1997) Int. Rev. Immunol., 16, 57-85). In other embodiments, the substitution mutation includes the “LALA-PG” Fc mutant with substitution of residues L234 and L235 to alanine and the P329 to glycine (see Brünker, P., et al. (2016) Mol. Cancer Ther).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001)).

In some embodiments, alterations are made in the Fc region that result in altered (i.e., either improved or diminished) C1q binding and/or Complement Dependent Cytotoxicity (CDC), for example, as described in U.S. Pat. No. 6,194,551, WO 99/51642, and Idusogie et al., J. Immunol. 164: 4178-4184 (2000).

Bispecific antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

The invention also provides immunoconjugates comprising a bispecific antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which a bispecific antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises a bispecific antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another embodiment, an immunoconjugate comprises a bispecific antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

In another embodiment, an immunoconjugate comprises a bispecific antibody as described herein conjugated to a non-cytotoxic agent, such as, for example, the artemisinins, such as artusenate, or cannabinoids, or naltrexone, or aspirin, or statins, or metabolic agents, such as metformin, doxycycline and anthelmintic.

Conjugates of a bispecific antibody of the invention and cytotoxic or non-cytotoxic agents may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to, such conjugates prepared with cross-linker reagents including, but not limited to, BMPS, EMCS, GMBS, HBVS, LC-SMCC, MBS, MPBH, SBAP, SIA, STAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

Any of the bispecific antibodies described herein may be produced using recombinant methods and compositions, for example, as described in U.S. Pat. No. 4,816,567. In one embodiment, an isolated nucleic acid encoding a bispecific antibody described herein is provided. Such a nucleic acid may encode an amino acid sequence comprising the VL and/or an amino acid sequence comprising the VH of each binding end of the bispecific antibody (e.g., the light and/or heavy chains of the antibody). In a further embodiment, one or more vectors (e.g., expression vectors) comprising such a nucleic acid are provided. In a further embodiment, a host cell comprising such a nucleic acid is provided. In one such embodiment, a host cell comprises (e.g., has been transformed with): (1) a vector comprising a nucleic acid that encodes an amino acid sequence comprising the VL of the bispecific antibody and an amino acid sequence comprising the VH of the bispecific antibody, or (2) a first vector comprising a nucleic acid that encodes an amino acid sequence comprising the VL of the bispecific antibody and a second vector comprising a nucleic acid that encodes an amino acid sequence comprising the VH of the bispecific antibody. In one embodiment, the host cell is eukaryotic, for example, a Chinese Hamster Ovary (CHO) cell or lymphoid cell (e.g., YO, NSO, Sp20 cell). In one embodiment, a method of making a bispecific antibody is provided, wherein the method comprises culturing a host cell comprising a nucleic acid encoding the bispecific antibody, as provided above, under conditions suitable for expression of the antibody, and optionally recovering the bispecific antibody from the host cell (or host cell culture medium).

For recombinant production of a bispecific antibody, nucleic acid encoding an antibody, for example, as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, bispecific antibodies may be produced in bacteria, in particular when glycosylation and Fc effector function are not needed. For expression of antibody fragments and polypeptides in bacteria, see, for example, U.S. Pat. Nos. 5,648,237, 5,789,199, and 5,840,523. See also Charlton, Methods in Molecular Biology, Vol. 248 (B. K. C. Lo, ed., Humana Press, Totowa, N.J., 2003), pp. 245-254, describing expression of antibody fragments in E. coli. After expression, the bispecific antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of a bispecific antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, for example, U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as YO, NSO and Sp2/0. For a review of certain mammalian host cell lines suitable for antibody production, see, for example, Yazaki and Wu, Methods in Molecular Biology, Vol. 248 (B. K. C. Lo, ed., Humana Press, Totowa, N.J.), pp. 255-268 (2003).

The bispecific antibodies provided herein may be identified, screened for, or characterized for their physical/chemical properties and/or biological activities by various assays known in the art and described herein in the Examples and throughout the specification.

In one aspect, a bispecific antibody of the invention is tested for both its antigen binding activity to Tie2 and to VEGF, e.g., by known methods such as ELISA, Western blot, surface plasmon resonance assays (e.g., BIACORE™), etc.

In one aspect, antigen binding activity (e.g., as indicated by KD) is measured using a BIACORE™ surface plasmon resonance (SPR) assay. For example, an assay using a BIACORE™-2000 or a BIACORE™-3000 (BIAcore, Inc., Piscataway, N.J.) is performed at 25° C. with immobilized antigen CM5 chips at about 10 response units (RU). In one embodiment, carboxymethylated dextran biosensor chips (CM5, BIAcore, Inc.) are activated with N-ethyl-N′-(3-dimethylaminopropyl)-carbodiimide hydrochloride (EDC) and N-hydroxysuccinimide (NHS) according to the supplier's instructions. Either Tie2 or VEGF antigen is diluted with 10 mM sodium acetate, pH 4.8, to 5 ug/ml (about 0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN™-20) surfactant (PBST) at 25° C. at a flow rate of approximately 25 μl/min. Association rates (kₒₙ) and dissociation rates (k_{off}) are calculated using a simple one-to-one Langmuir binding model (BIACORE™ Evaluation Software version 3.2) by simultaneously fitting the association and dissociation sensorgrams. The equilibrium dissociation constant (KD) is calculated as the ratio k_{off}/kₒₙ. See, for example, Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette. KD may also be measured using a BIACORE™ SPR assay.

In another aspect, competition assays may be used to identify a bispecific antibody that competes with another antibody for binding to Tie2 and/or VEGF. In certain embodiments, such a competing antibody binds to the same epitope (e.g., a linear or a conformational epitope) that is bound by an antibody as described herein. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized Tie2 or VEGF is incubated in a solution comprising a first labeled antibody that binds to Tie2 or VEGF and a second unlabeled antibody that is being tested for its ability to compete with the first antibody for binding to Tie2 or VEGF. The second antibody may be present in a hybridoma supernatant. As a control, immobilized Tie2 or VEGF is incubated in a solution comprising the first labeled antibody but not the second unlabeled antibody. After incubation under conditions permissive for binding of the first antibody to Tie2 or VEGF, excess unbound antibody is removed, and the amount of label associated with immobilized Tie2 or VEGF is measured. If the amount of label associated with immobilized Tie2 or VEGF is substantially reduced in the test sample relative to the control sample, then that indicates that the second antibody is competing with the first antibody for binding to Tie2 or VEGF. See Harlow and Lane (1988) Antibodies: A Laboratory Manual ch. 14 (Cold Spring Harbor Laboratory, Cold Spring Harbor, N.Y.).

In one aspect, assays are provided for identifying bispecific antibodies thereof having biological activity. See the Examples section. Biological activity may include, for example, activating, agonizing, increasing, enhancing one or more biological activities of Tie2 or VEGF. Bispecific antibodies having such biological activity in vivo and/or in vitro are also provided herein, see Table 1.

In certain embodiments, labeled bispecific antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like. In another embodiment of the invention, the bispecific antibody need not be labeled, and the presence thereof can be detected using a labeled bispecific antibody which binds to the bispecific antibody, such as, for example, an anti-horseradish peroxidase antibody, well-known in the art.

The bispecific antibodies of the present invention may be employed in any known assay method, such as competitive binding assays, direct and indirect sandwich assays, and immunoprecipitation assays. Zola et al., Monoclonal Antibodies: A Manual of Techniques, pp. 147-158 (CRC Press, Inc. 1987).

Competitive binding assays rely on the ability of a labeled standard to compete with the test sample analyze for binding with a limited amount of antibody. The amount of antigen in the test sample is inversely proportional to the amount of standard that becomes bound to the antibodies. To facilitate determining the amount of standard that becomes bound, the antibodies generally are insolubilized before or after the competition, so that the standard and analyze that are bound to the antibodies may conveniently be separated from the standard and analyze which remain unbound.

Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three-part complex. See, for example, U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

### Pharmaceutical Formulations

Therapeutic formulations of the bispecific antibody of the invention or immunoconjugates of the invention or fusion polypeptides of the invention provided herein may be prepared for storage as lyophilized formulations or aqueous solutions by mixing the polypeptide having the desired degree of purity with optional “pharmaceutically-acceptable” carriers, excipients, or stabilizers typically employed in the art (all of which are termed “excipients”). For example, buffering agents, stabilizing agents, preservatives, isotonifiers, non-ionic detergents, antioxidants and other miscellaneous additives. See e.g., Remington's Pharmaceutical Sciences, 16^{th} edition, A. Osol, Ed. (1980). Such additives must be nontoxic to the recipients at the dosages and concentrations employed.

### Therapeutic Methods

The bispecific antibodies of the invention provided herein may be used in therapeutic methods for treating, preventing, mitigating, diagnosing various diseases, including, without limitation, ocular diseases, ischemic diseases or injuries, and infectious diseases as defined herein.

In one embodiment, the bispecific antibodies of the invention provided herein may be used to treat, for example, diabetic retinopathy (DR), diabetic macular edema (DME), proliferative diabetic retinopathy (PDR) whether non-proliferative or proliferative, age-related macular degeneration (AMD) whether wet or dry, geographic atrophy (GA), retinopathy of prematurity (ROP), glaucoma, retinal vein occlusion (RVO), central RVO, or branched RVO and the like.

In another embodiment the bispecific antibodies of the invention provided herein may be used to treat, for example, sepsis, dengue virus infection, tuberculosis, coronavirus infection or influenza and the like.

In yet another embodiment, the bispecific antibodies of the invention provided herein may be used to treat, for example, diabetic nephropathy, acute kidney injury, chronic kidney disease, kidney or other organ transplantation and the like.

### EXAMPLES

The following are examples of methods and the bispecific antibody constructs of the invention. It is understood that various other embodiments may be practiced, given the general description provided above.

### Example 1: Tie2/VEGF Bispecifics Activate the Akt Pathway In Vitro

This is a cell-based assay used to determine the levels of phosphorylated Akt protein in HUVEC cells. This determines whether the bispecific antibody constructs of the invention are able to activate the correct pathway in vitro.

HUVEC cell treatment in a 96 well plate: HUVEC cells from a single donor were cultured in endothelial cell growth media with supplements in a 37° C. incubator at 5% C02 and 3% oxygen. One day before the assay, cells were plated at 80,000 cells per well in a 96 well tissue culture plate. On the following day, cells were starved for 4 hrs in non-supplemental endothelial basal media. Post starvation of the cells, non-supplemented endothelial basal media containing either a bispecific antibody construct or angiopoietin-2 (a Tie2 ligand) at a concentration range of 100 nM to 0.14 nM were added to the cells. Cells were then incubated in a 37° C. incubator at 5% C02 and 3% oxygen for 20 min. After the incubation, media from the 96 well plate was removed and stored in the −80° C. freezer.

The MSD ELISA assay: MSD-TRIS 5% (w/v) blocking buffer was added to streptavidin coated small spot 96 well MSD plates at a volume of 150 uL per well and incubated on an orbital shaker at 600 rpm for 1 hr at room temperature (RT). Immediately after the blocking step, the plate was washed once with 150 μL of the MSD-TRIS wash buffer. Biotinylated anti-pAkt (s473) capture antibody at a concentration of 100 ng/well was added to the plate and incubated at RT for 2 hrs. Previously treated HUVEC 96 well cell plates were removed from the −80° C. freezer and 65 μL of the MSD-TRIS lysis buffer with protease and phosphatase inhibitors was added to each well. After adding the lysis buffer, the plate was incubated on a shaker at 600 rpm for 1 hr at RT. After 2 hr incubation with the capture antibody, the plate was washed 3 times with 150 ul of the MSD-TRIS wash buffer. Cell lysates were added at 50 ul per well and plates were incubated on a shaker at 600 rpm at 4° C. for overnight. Following incubation, plates were washed 3 times with 150 ul of MSD-TRIS wash buffer, sulfo-tagged anti-Akt antibody was added to each well at a concentration of 200 ng/well and incubated at room temperature for 2 hrs on a shaker at 600 rpm. Post-incubation, plates were washed 3 times with 150 ul of the MSD-TRIS wash buffer, and 150 μL of the 2× diluted concentrated MSD read buffer was added to each well (the original stock concentration is 4×). The plate was read on the MSD S-600 instrument.

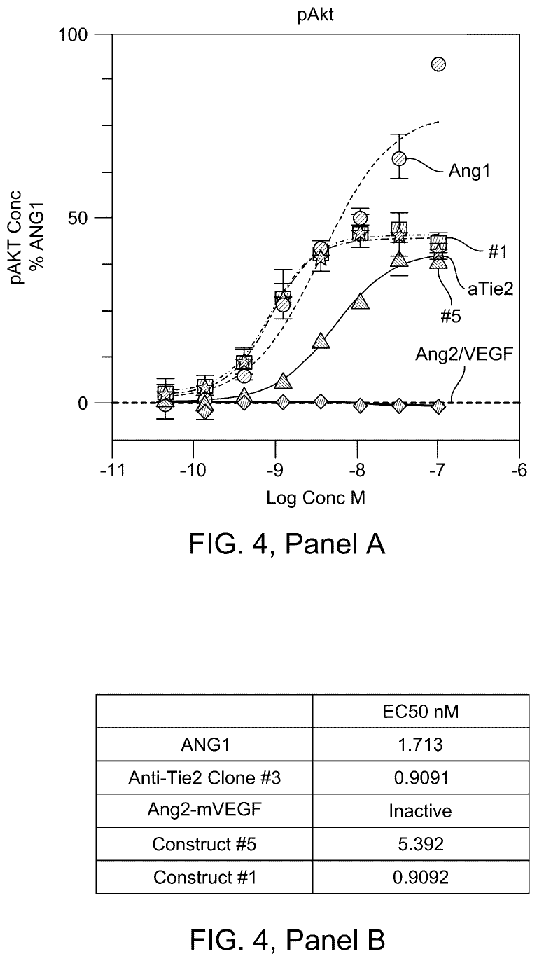

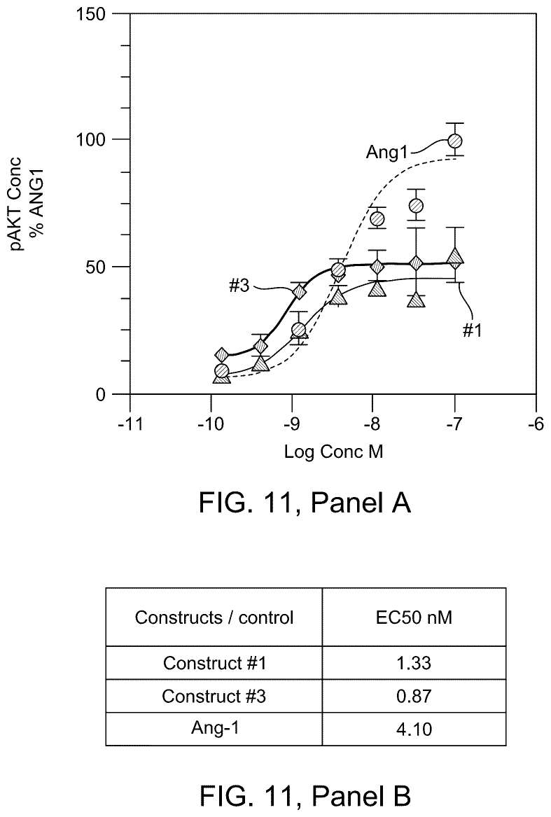

Electrochemiluminescence signal was determined. The signal was normalized by deducting the background signal from the cells treated with non-supplemented endothelial basal media. Normalized signal from the cells treated with various concentrations of the drug or angiopoietin was fitted to the “log(agonist) vs normalized response-variable slope” equation in GraphPad Prism. The potencies were reported as EC50. See FIG. 4, panels A-B, and FIG. 11, panels A-B.

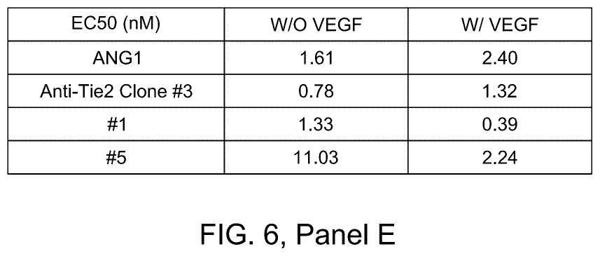

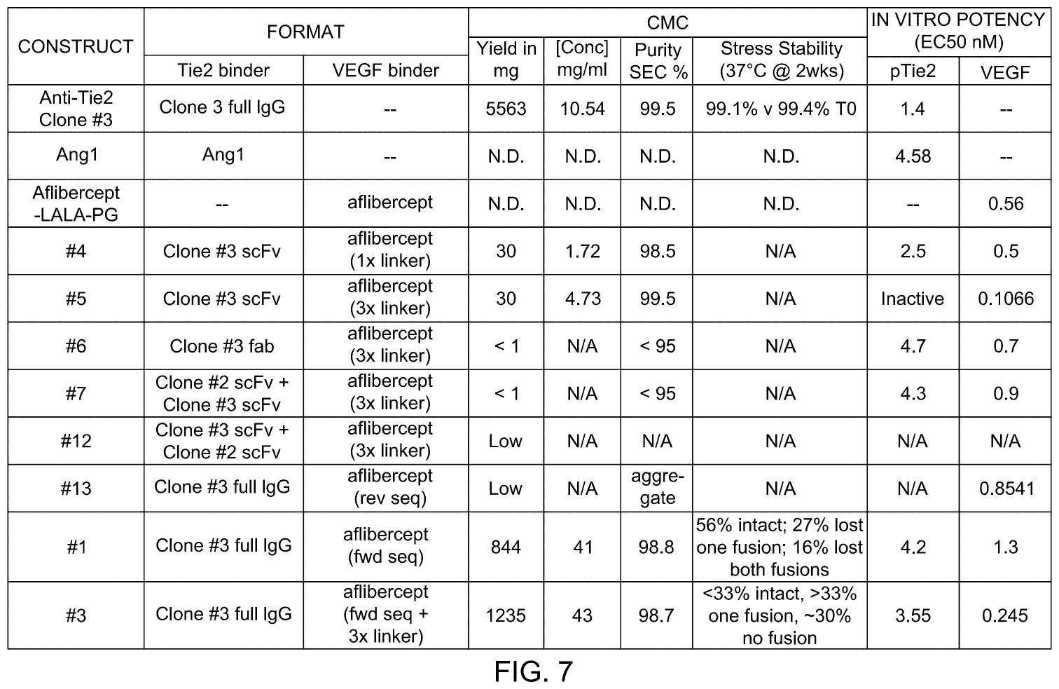

The results shows that Construct #1 activates downstream AKT signaling similar to the Anti-Tie2 Clone #3. Potency of Construct #1 is approximately 5-fold higher than Construct #5. See also FIG. 7. In contrast, the Ang2-mVEGF bispecific which is a faricimab-like bispecific which is an Ang2-dependent Tie2 activator, does not activate the AKT downstream pathway. Similarly, in a separate experiment, Constructs #1 and #3 both activate the downstream AKT pathway in contrast to the Ang1 control (FIG. 11, panels A-B).

### Example 2: Determining Tie2 Potency In Vitro

A pTie2 cell-based assay was been developed to determine the levels of phosphorylated Tie2 protein in human umbilical vein endothelial cells (HUVEC). Using this assay, the potencies of various bispecific antibody constructs were tested and quantitated to enable a rank-ordering of bispecific constructs of the invention.

HUVEC cell treatment in a 96 well plate: HUVEC cells from a single donor have been used in all assays. Cells were cultured in endothelial cell growth media with supplements in a 37° C. incubator at 5% C02 and 3% oxygen. One day before the assay, cells were plated at 80,000 cells per well in a 96 well tissue culture plate. On the following day, cells were starved for 4 hrs in non-supplemental endothelial basal media. Post-starvation of the cells, non-supplemental endothelial basal media containing either bispecific antibody constructs or Angiopoietin-2 (a Tie2 ligand) at a concentration range of 100 nM to 0.14 nM were added to cells. Cells were then incubated in a 37° C. incubator at 5% C02 and 3% oxygen for 20 min. After the incubation, the media from the 96 well plate was removed, and the plates were stored in a −80° C. freezer.

Meso Scale Diagnostics (MSD) ELISA assay: To determine levels of phospho-Tie2 present in treated cells, an MSD ELISA assay was employed. MSD-TRIS 5% (w/v) blocking buffer was added to the streptavidin-coated small spot 96 well MSD plates at a volume of 150 uL per well and incubated on an orbital shaker at 600 rpm for 1 hr at RT. Immediately after the blocking step, plates were washed once with 150 μL of MSD-TRIS wash buffer. Biotinylated anti-Tie2 capture antibody at a concentration of 50 ng/well was added to plates, which were incubated at room temperature for 2 hrs. Previously treated HUVEC 96 well cell plates were removed from the −80° C. freezer and 65 μL of the MSD-TRIS lysis buffer with protease and phosphatase inhibitors was added to each well. After adding lysis buffer, plates were incubated on a shaker at 600 rpm for 1 hr at RT. A 3-fold serial dilution of standard recombinant human pTie2 protein was made at a concentration range of 25 ng/mL to 0.39 ng/mL in the MSD-TRIS lysis buffer with protease and phosphatase inhibitors. After a 2 hr incubation with the capture antibody, plates were washed 3 times with 150 μl of MSD-TRIS wash buffer. Standards or cell lysates were added at 50 ul per well and plates were incubated on a shaker at 600 rpm at 4° C. overnight. Following the incubation, plates were washed 3 times with 150 ul of MSD-TRIS wash buffer, and sulfo-tagged anti-phospho-tyrosine detection antibody was added to each well at a concentration of 100 ng/well, then plates were incubated at room temperature for 2 hrs on a shaker at 600 rpm. Post-incubation, plates were washed 3 times with 150 ul of MSD-TRIS wash buffer, and 150 μL of the 2× concentrated MSD read buffer was added to each well (the original stock concentration is 4×). The plate was read on the MSD S-600 instrument.

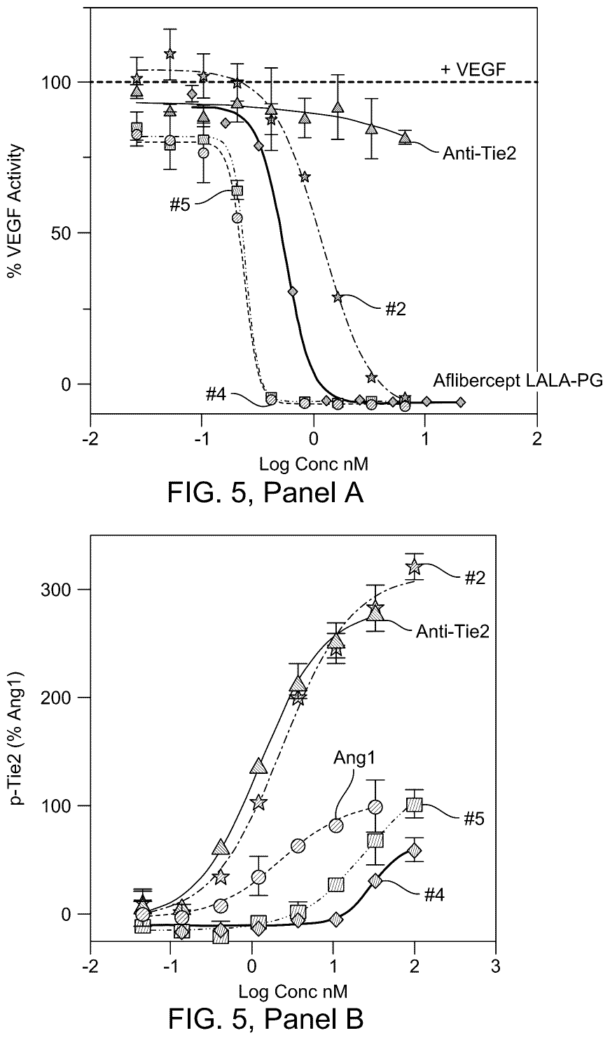

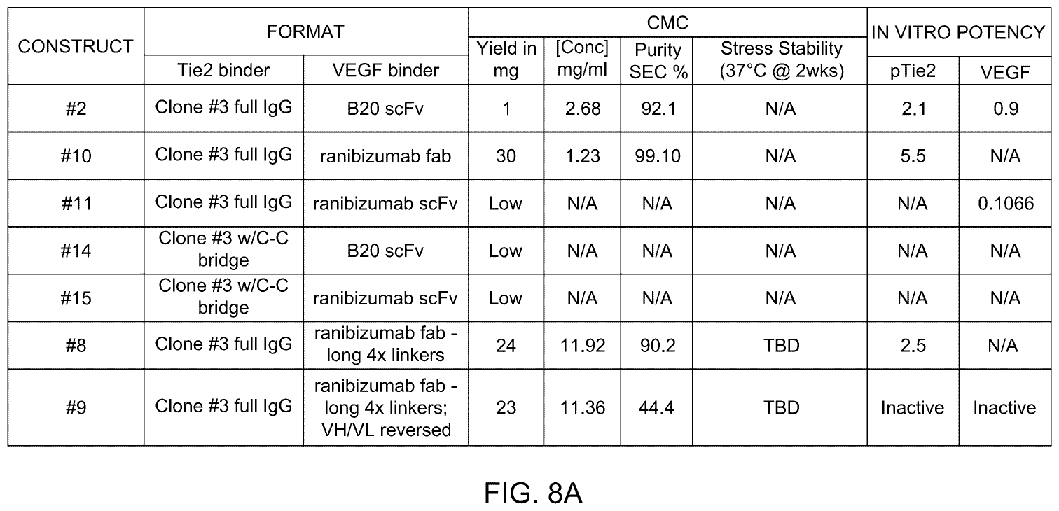

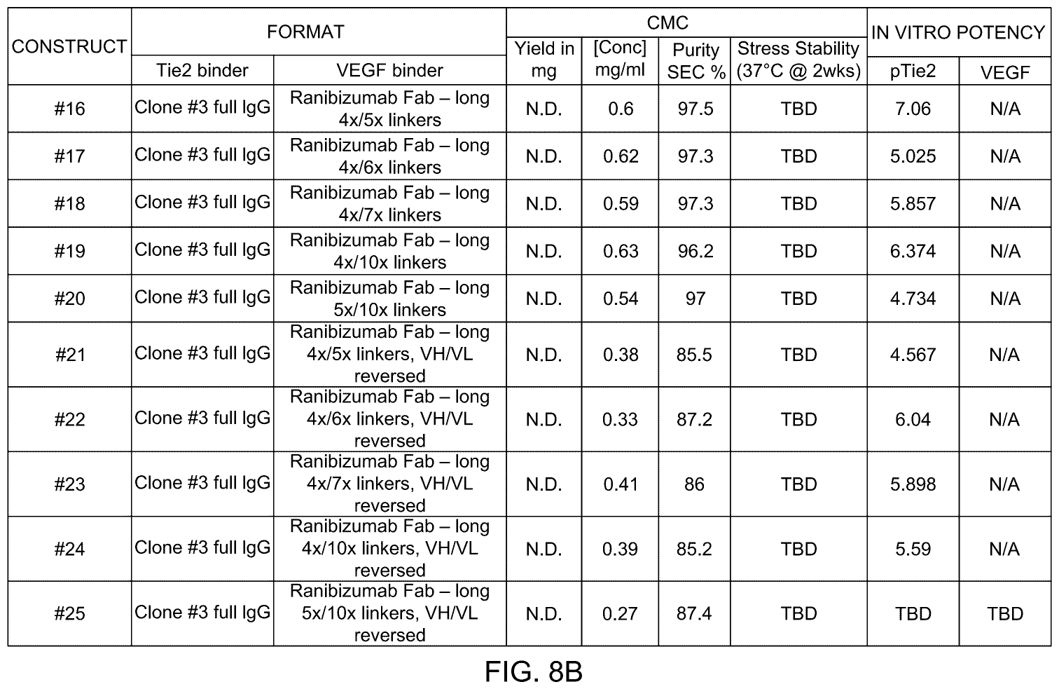

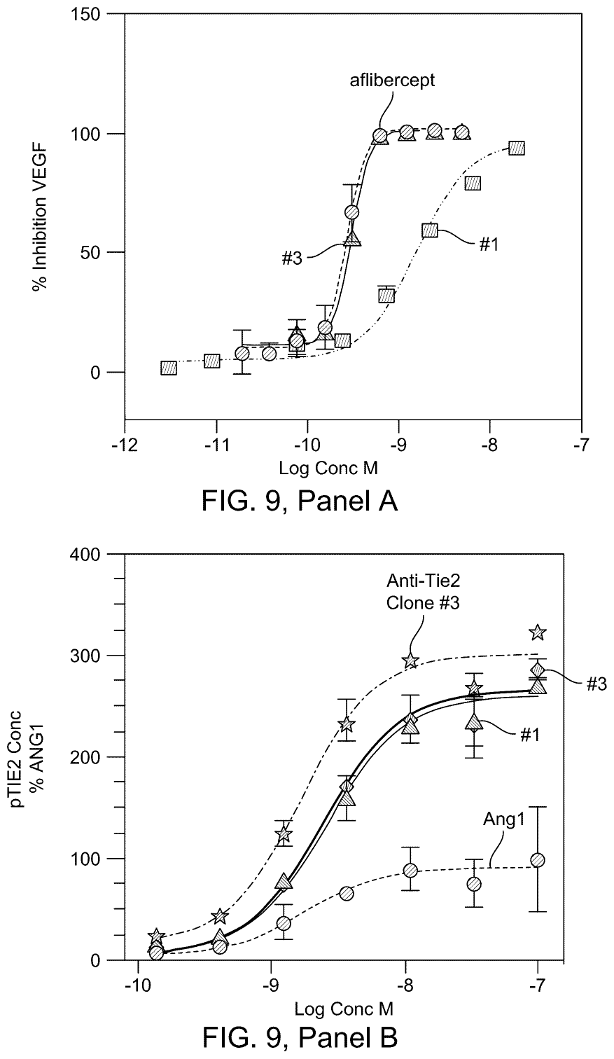

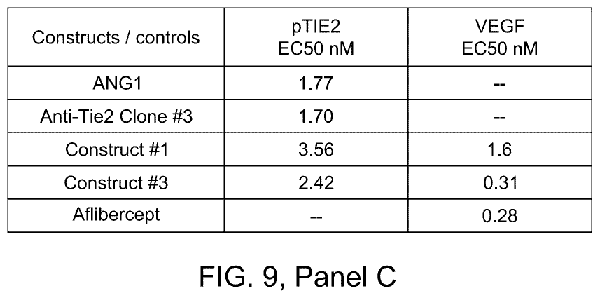

Electrochemiluminescence signal was analyzed using Discovery Workbench™ software. Concentration of pTie2 in HUVEC cells that were treated with various concentrations of bispecific constructs tested (Constructs #1, #2, #3, #4 or #5) or controls (anti-Tie2 Clone #3 or Ang1) were determined by four-parameter fit logistic regression analysis based on the standard curve. Normalized signals were plotted against the concentrations and fitted to the “log(agonist) vs normalized response-variable slope” equation in GraphPad Prism. The potencies were reported as EC50 nM. See FIG. 5, panel B, FIG. 7, FIGS. 8A-8B, FIG. 9, panel B and FIG. 9, panel C.

Different bispecific antibody constructs demonstrate different measured in vitro p-Tie2 EC50 values which underscores the unpredictability of a particular construct with respect to its p-Tie2 in vitro potency.

### Example 3: Determining VEGF Potency In Vitro

The VEGF Bioassay from Promega corporation is a luminescent cell-based assay that measures VEGF inhibition via KDR/VEGFR2. The KDR/NFAT-RE HEK293 Cells have been engineered to express the NFAT response element upstream of Luc2P as well as exogenous KDR. Bioluminescent signal is detected and quantified using Bio-Glo™ Luciferase Assay System. This assay has been used to determine the in vitro VEGF potencies of various bispecific antibody constructs.

KDR/NFAT-RE HEK293 Cell treatment in a 96 well plate: Frozen cells were thawed and resuspended in pre-warmed assay buffer (DMEM media with 10% fetal bovine serum). Cells were plated at 5000 cells per well in a 96 well tissue culture plate. Serial dilutions of bispecific antibody constructs tested (Constructs #1, #2, #3, #4 or #5) or controls (aflibercept or aflibercept-LALA-PG or anti-Tie2 Clone #3) were added to the plates, at a final concentration range of 20 nM to 1 μM, or bevacizumab at a final concentration range of 40 nM to 2 μM, in duplicate. This step was followed by addition of 0.5 nM human recombinant VEGF-165 protein in the case of FIG. 5. Negative controls included in each assay were cells treated with only 0.5 nM VEGF-165. Plates were incubated in a 37° C., 5% C02 humidified incubator for 6 hours. Post-incubation, 75 μl of Bio-Glo™ Reagent was added to all wells and plates were incubated at room temperature for 10 min. Post-incubation, the luminescence was measured using Enspire™ luminescence plate reader. The percent inhibition was calculated based on VEGF-165 treated cells as 0% inhibition. The percent inhibition was plotted against the concentrations and fitted to the “log(inhibitor) vs normalized response-variable slope” equation in GraphPad Prism and the potencies were reported in EC50 nM. See FIG. 5, panel A, FIG. 7, FIGS. 8A-8B, FIG. 9, panel A and FIG. 9, panel C.

The results suggest that different bispecific constructs can demonstrate varying VEGF inhibition potencies.

### Example 4: Determining In Vitro Phospho-Tie2 Potency in HUVECs in the Presence of Recombinant Human VEGF-165

The pTie2 cell-based assay was developed to determine the levels of phosphorylated Tie2 protein in HUVECs to investigate the effect of VEGF on the bispecific antibody constructs in activating the Tie2 pathway. Potencies were determined both in the presence and absence of VEGF.

HUVEC cell treatment in a 96 well plate: HUVECs were cultured in the endothelial cell growth media with the supplements in a 37° C. incubator at 5% C02 and 3% oxygen. A day before the assay, the cells were plated at 80,000 cells per well in a 96 well tissue culture plate. On the following day, cells were starved for 4 hrs in non supplemental endothelial basal media. Post starvation of the cells, the cells were treated with or without 20 nM (final concentration) VEGF-165 recombinant protein in the non supplemental endothelial basal media for 5 min. Post VEGF treatment, the bispecific antibody constructs or Angiopoietin-2 were added to the cells at a concentration range of 100 nM to 0.14 nM. The cells were then incubated in a 37° C. incubator at 5% C02 and 3% oxygen for 20 min, and after the incubation, the media from the 96 well plate was removed and the plates were stored in the −80° C. ultra-low freezer.

The MSD ELISA assay: This assay was performed in a streptavidin coated small spot 96 well MSD plate. The MSD-TRIS 5% (w/v) blocking buffer, was added to the plate and at a volume of 150 μL per well and incubated on an orbital shaker at 600 rpm for 1 hr at RT. Immediately after the blocking step, the plate was washed once with 150 μL of the MSD-TRIS wash buffer. The biotinylated anti-Tie2 capture antibody at a concentration of 50 ng/well was added to the plate, and incubated at RT for 2 hrs. Previously treated HUVEC 96 well cell plate was removed from the −80° C. ultra low freezer, and 65 μL of the MSD-TRIS lysis buffer with protease and phosphatase inhibitors was added to each well. After adding the lysis buffer, the plate was incubated on a shaker at 600 rpm for 1 hr at RT. A 3-fold serial dilution of the standard recombinant human pTie2 protein was made at a concentration range of 25 ng/mL to 0.39 ng/mL in the MSD-TRIS lysis buffer with protease and phosphatase inhibitors. After the 2 hr incubation with the capture antibody, wash the plate 3 times with 150 ul of the MSD-TRIS wash buffer. The standards or the cell lysates were added at 50 ul per well and the plate was incubated on a shaker at 600 rpm at 4° C. overnight. Following the incubation, the plates were washed 3 times with 150 μl of the MSD-TRIS wash buffer, and the sulfo-tagged anti-pan phospho-tyrosine detection antibody was added to each well at a concentration of 100 ng/well and incubated at room temperature for 2 hrs on a shaker at 600 rpm. Post incubation the plates were washed 3 times with 150 ul of the MSD-TRIS wash buffer, and 150 μL of the 2× dilution of the concentrated MSD read buffer was added to each well (the original stock concentration is 4×). The plate was read on the MSD S-600 instrument.

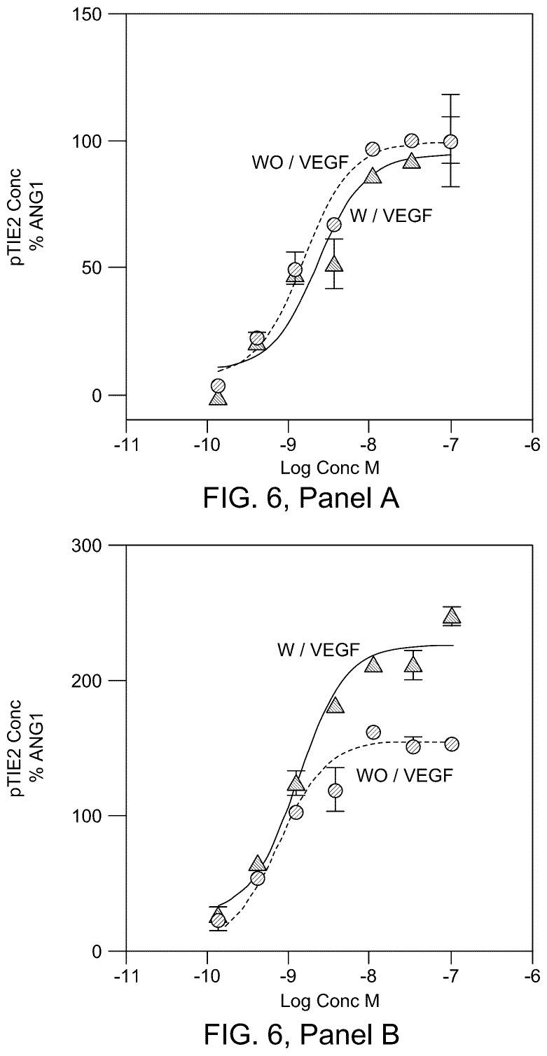

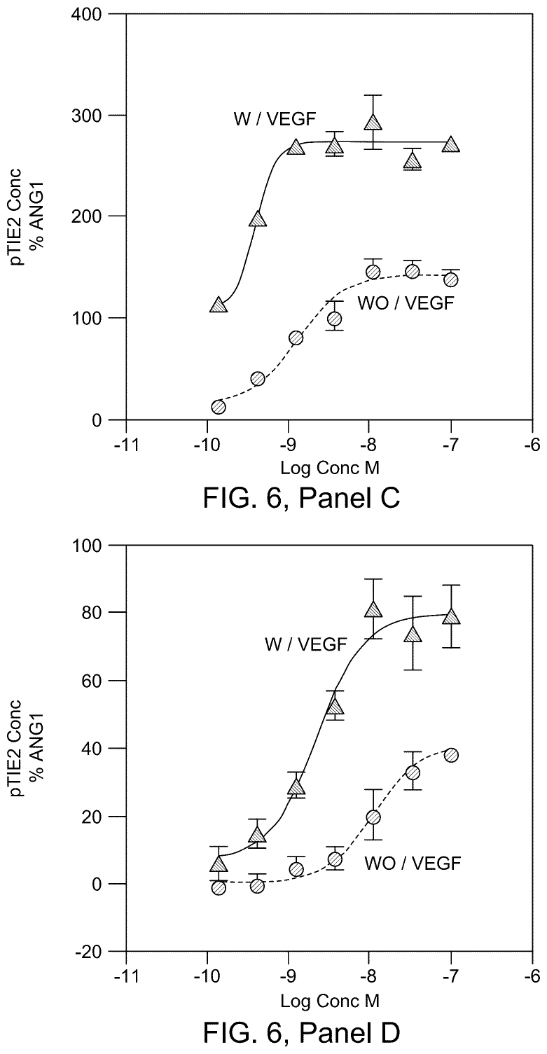

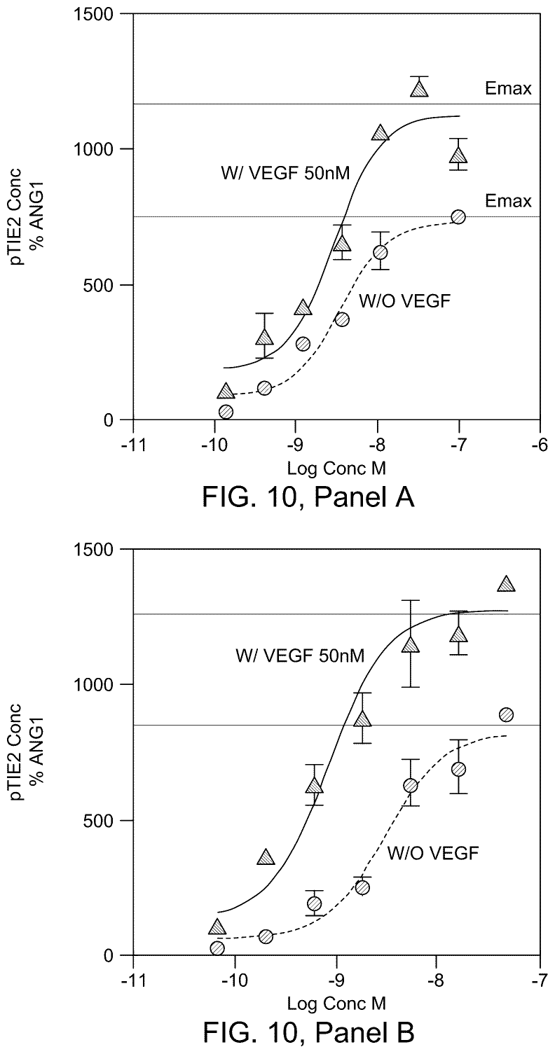

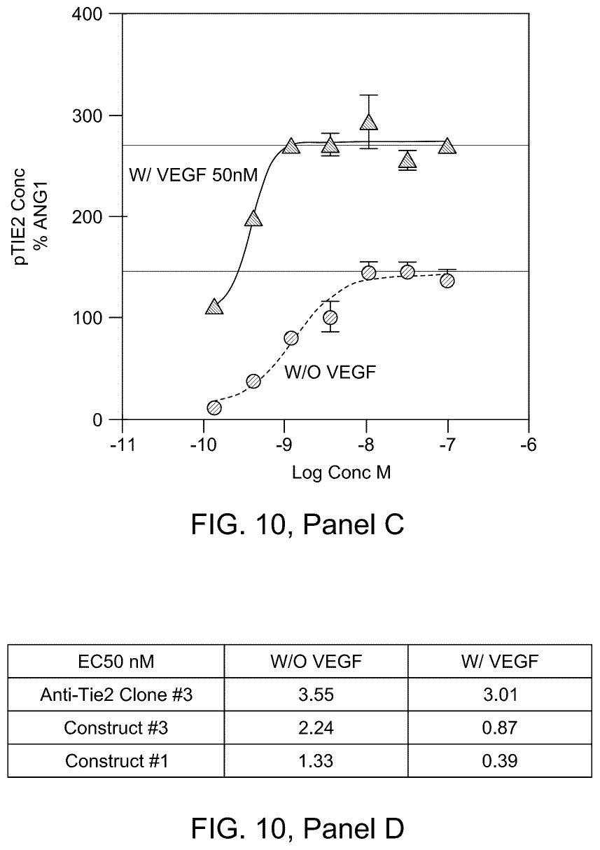

The readout from the plate is the electrochemiluminescence signal, which was analyzed using the discovery work bench software. The concentration of the pTie2 in the HUVEC cells that were treated with various concentrations of the bispecific antibody constructs (Constructs #1, #3, or Ang1 or the anti-Tie2 Clone #3 were determined by four-parameter fit logistic regression analysis based on the standard curve. Normalized signals were plotted against the concentrations and fitted to the “log(agonist) vs normalized response-variable slope” equation in GraphPad Prism. The potencies were reported as EC50 nM. See FIG. 6, panels A-E and FIG. 10, panels A-D.

The results suggest that the bispecific antibody constructs tested have an improved in vitro Tie2 potency in the presence of VEGF (FIG. 6, panel E and FIG. 10, panel D), while the Ang1 and the anti-Tie2 Clone #3 tested are not significantly affected by VEGF (FIG. 6, panel A and FIG. 6, panel E and FIG. 10, panel A and FIG. 10, panel D). This is a surprising result and suggests that in vivo, the endogenous presence of VEGF in the microenvironment of a diseased tissue may enhance the Tie2 activity of certain bispecific antibody constructs of the invention.

### Example 5: Measuring Tie2/VEGF Bispecific Yield, Purity and Stability

Important to the success of generating a viable biologic drug product is the manufacturability of the product. In view of the unpredictability of bispecific antibodies in this regard, certain CMC criteria was used to determine which bispecific antibody of the invention would show the most promise in terms of each molecule's (a) titer; (b) yield; (c) purity; and (d) stability.

Plasmid Preparation: The gene sequence for each specific bispecific construct was amplified by PCR and inserted into a corresponding expression vector. The sequence of the constructed vector was validated by sequencing. The correct plasmid for each bispecific antibody construct was amplified and transmitted to downstream.

Transient Transfection and Cell Culture: For transient transfection, the plasmids were mixed with transfection reagents at an optimal ratio and then added into the flask using a transient CHO-K1 or HEK293 cell line. The cells were cultured in a serum-free medium and maintained in Erlenmeyer Flasks on an orbital shaker or the bioreactor by a suitable stirring speed at 37° C. for 6 days.

Purification and Analysis: Standard purification procedures were employed: (a) the cell culture broth for each bispecific antibody construct was centrifuged; (b) the cell culture supernatant was purified using Protein A affinity chromatography and then gel filtration affinity purification; and (c) the purified bispecific antibody was analyzed for endotoxin burden, by SDS-PAGE, and SEC-HPLC.

Measurement of titer or yield: Titer, in biologics production, is the concentration of the total product in the production media, as measured by certain analytical methods (ELISA or HPLC) using a standard curve of reference material. Yield refers to the total mass of the final, purified and processed material or drug substance. Intermediate yield is the mass of the total product recovered at various stages of product harvesting and processing. Yields are calculated using the formula [conc]*volume, and these numbers are specific to each step/intermedia/product. Intermediate yield will be higher than final yield, because while impurities are being removed, intrinsic product loss is also unavoidable. Concentration [conc] is the strength of the DS protein in its final formulation buffer. It can either determined experimentally or calculated using a known extinction coefficient and the A280 measurement. See FIGS. 7 and 8A-8B.

Purity Assessment by SEC-HPLC: Size exclusion HPLC (SEC-HPLC) separates proteins on the basis of hydrodynamic volume, which generally correlates with molecular weight. SEC resins have pores into which proteins can diffuse while passing through the column. The time proteins spend passing through the pores, which delays their elution from the column, is inversely proportional to their hydrodynamic volume. Thus, elution times in SEC are inversely proportional, to a first approximation, to molecular weight. When SEC is employed to assess the quality/purity of a protein sample, often times, besides the main product peak, there may be higher order species (or High Molecular Weight, HMW) eluted at the shorter retention time that result from aggregation or multimerization of the product, and/or degradation/truncation products (LMW) that elute later than the main product peak. Both HMW and LMW species are normally considered product related impurities. There can also be a small likelihood of impurities from the process or raw materials. For biologics, in general, >98% main peak and <2% HMW in DS as the initial SEC purity criteria would be acceptable. See FIGS. 7 and 8A-8B.

Stress Stability Test: Each lot of bispecific antibody construct produced and purified as described above was aliquoted into 8 or more equal fractions. Half of the aliquots were stored at 37° C. for two weeks, while the other half was stored at 5° C. or was frozen as “control time zero” or (TO). At the end of the stress test, the 37° C. samples were compared to the TO samples using SEC-HPLC and p-Tie2 and VEGF potency assays, as described in Examples 2 and 3 above. See FIGS. 7 and 8A-8B.

The bispecific antibody constructs tested and results shown in FIGS. 7 and 8A-8B showed different CMC characteristics, suggesting that not all Tie2/VEGF bispecific antibody constructs generated would be suitable for further development from a CMC perspective as a possible drug candidate.

### Example 6: Evaluating Target Engagement (TE) of Tie2/VEGF Bispecifics in an Oxygen-Induced Retinopathy (OIR) Mouse Model

The OIR model is based on the exposure of mouse pups to hyperoxia during a phase when their retinal vasculature is still developing. This leads to capillary depletion, and upon return to room air, results in retinal ischemia and proliferative vascular disease in the retinal vasculature or oxygen-induced retinopathy. The goals of the experiments were to: first, evaluate the ability for the bispecific molecules Construct #1 and Construct #3 to engage its target protein, Tie2 receptor (TE: target engagement), following intravitreal (IVT) delivery to hyperoxic pups in comparison to a faricimab murine surrogate or “FMS”, which is a bispecific construct containing the known anti-mouse VEGF clone B20 (see Liang, W C, et al., JBC, 2006: 281, 951-961) and the known anti-Ang2 clone LC10 (Roche R05485202); second, evaluate the Ang2 neutralisation of the bispecific molecules Construct #1 and Construct #3 in comparison to the FMS following IVT delivery in hyperoxic pups.

C57BL/6J pups at postnatal day 7 (P7) were housed in a hyperoxic chamber (75% 02) for 5 days (n=10 per cage) leading to vessel regression in the center of the retina. CD-1 fostering mothers were rotated before and 2-3 days after entering the chamber. At P12, pups were returned to room air where the relative hypoxia triggers abnormal neovascularization, then they were dosed intravitreally with bispecific constructs of the invention or a negative control, as described below.

At P17, all groups, including naïve OIR mice were euthanized. Eyes were enucleated and fixed in 4% paraformaldehyde for 1 hour.

Retinas were dissected and incubated overnight with rhodamine-labeled lectin from Bandeiraea simplicifolia (Griffonia simplicifolia) (1:100) in 1 mM CaCl₂) in PBS to visualize vaso-obliterated (VO) or neovascular (NV) areas. Stained retinas were flat mounted onto slides and imaged on the Zeiss® AxioScan. Images were analyzed on Visiopharm® to determine % VO or % NV of total retina. All data was expressed as the mean±SD where each data point represents an average of both retinas from one pup. Statistical significance of the bispecific constructs tested was determined using an ordinary one-way ANOVA followed by Dunnett's multiple comparison test against the NSIgG group at the respective timepoints with Prism 8 software from GraphPad® (San Diego, CA, USA) where statistical significance was determined to be p<0.05.

Alternatively, harvested retinas may be assessed by the Meso Scale Diagnostics (MSD) ELISA assay as described above in Example 2 in order to determine levels of phospho-Tie2 present in retinal tissues of treated OIR mice. MSD-TRIS 5% (w/v) blocking buffer was added to the streptavidin-coated small spot 96 well MSD plates at a volume of 150 uL per well and incubated on an orbital shaker at 600 rpm for 1 hr at RT. Immediately after the blocking step, plates were washed once with 150 μL of MSD-TRIS wash buffer. Biotinylated anti-Tie2 capture antibody at a concentration of 50 ng/well was added to plates, which were incubated at room temperature for 2 hrs. Previously treated HUVEC 96 well cell plates were removed from the −80° C. freezer and 65 μL of the MSD-TRIS lysis buffer with protease and phosphatase inhibitors was added to each well. After adding lysis buffer, plates were incubated on a shaker at 600 rpm for 1 hr at RT. A 3-fold serial dilution of standard recombinant human pTie2 protein was made at a concentration range of 25 ng/mL to 0.39 ng/mL in the MSD-TRIS lysis buffer with protease and phosphatase inhibitors. After a 2 hr incubation with the capture antibody, plates were washed 3 times with 150 μl of MSD-TRIS wash buffer. Standards or cell lysates were added at 50 ul per well and plates were incubated on a shaker at 600 rpm at 4° C. overnight. Following the incubation, plates were washed 3 times with 150 ul of MSD-TRIS wash buffer, and sulfo-tagged anti-phospho-tyrosine detection antibody was added to each well at a concentration of 100 ng/well, then plates were incubated at room temperature for 2 hrs on a shaker at 600 rpm. Post-incubation, plates were washed 3 times with 150 ul of MSD-TRIS wash buffer, and 150 μL of the 2× concentrated MSD read buffer was added to each well (the original stock concentration is 4×). The plate was read on the MSD S-600 instrument.

This experiment was performed testing the following: 10 ug/eye each of bispecific Constructs #1, #3, an FMS control as a comparator, and an irrelevant negative control NSIgG. OIR mouse pups were dosed either at P13 and retinas harvested at P15 (48 hour collection) or dosed at P15 and retinas harvested at P16 (24 hour collection). Comparisons were made relative of the time matched NSIgG control group 10 ug/eye dose. One-way ANOVA with Dunnet's post hoc. **p<0.01; ****p<0.0001.

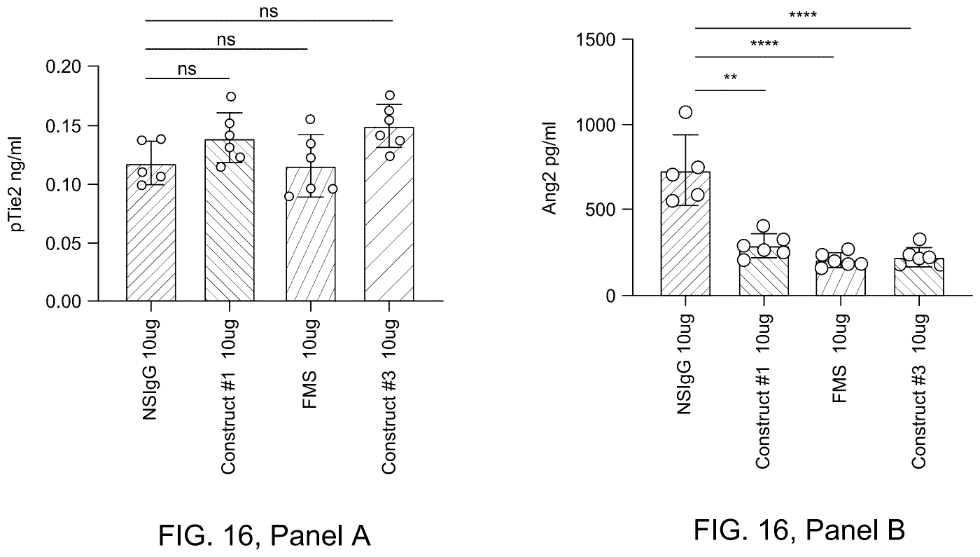

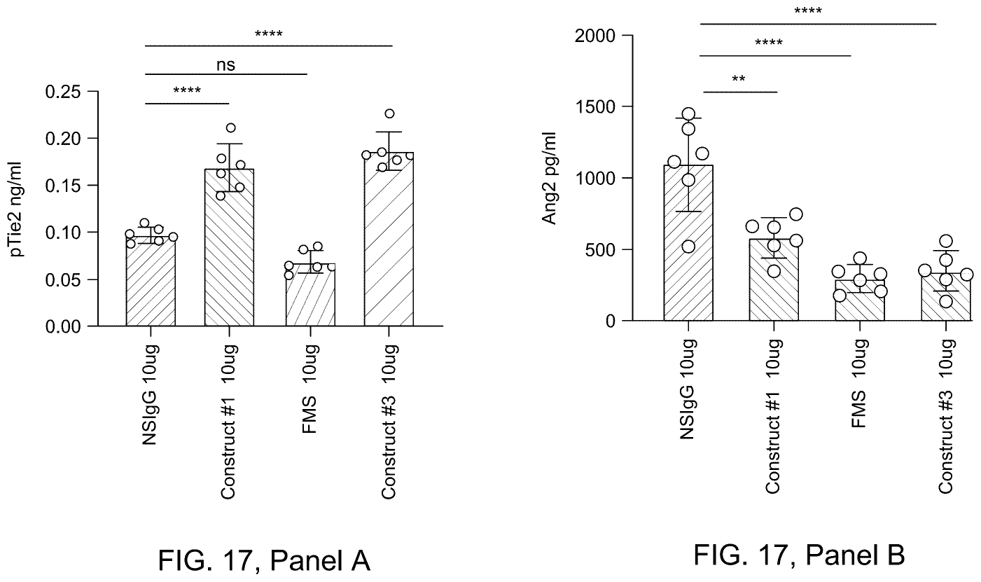

When dosed at P13 and harvested at 48 hr, Construct #3 (10 ug), Construct #1 (10 ug) and FMS (10 ug) did not show significant upregulation of pTie2 in comparison to the NSIgG control (10 ug) in the hyperoxic retina. See FIG. 16, panel A. However, all three molecules demonstrated significant Ang2 reduction. See FIG. 16, panel B. When dosed at P15 and harvested at 24 hr, Both Construct #3 (10 ug) and Construct #1 (10 ug) showed significant pTie2 upregulation compared to the NSIgG control (10 ug). See FIG. 17, panel A. However, this was not observed with FMS (10 ug). See FIG. 17, panel A. All three molecules significantly downregulated Ang2. See FIG. 17, panel B.

Constructs #1 and #3 both exhibited engagement of its target Tie2 receptor, significantly upregulating pTie2 and modulating Ang2 levels in the hyperoxic eye at P15. In contrast, FMS appeared to have neutralized Ang2 but did not show any change in pTie2 levels, possibly due to the inefficiency of the Ang1 protein at P15. At P13, all three molecules showed significant reduction of Ang2 while pTie2 signals might have been lost by 48 hr. This study showed the ability of the tested bispecific constructs of the invention to trigger pTie2 induction with modulation of the biomarker Ang2 in a disease state regardless of the Ang1 and Ang2 levels.

### Example 7: Efficacy of Tie2/VEGF Bispecifics in a Laser-Induced Choroidal Neovascularization (CNV) Rat Model

This study will be done to determine effect of certain bispecific antibody constructs on vascular leak and choroidal neovascularization in a rat laser CNV model. The laser-induced CNV rat model involves rupture of Bruch's membrane, leading to an inflammatory/wound-healing response and concomitant CNV, thereby mimicking wet or neovascular AMD. The choroidal capillaries are explicitly involved in the neovascular response, and this model produces a similar angiographic appearance to wet or neovascular AMD. The animal experiments are to be carried out by EyeCRO™ LLC (Oklahoma City, OK).

Animal Housing and Welfare: Female Brown Norway Rats, 9-12 weeks old and weighing 126-150 g are obtained for the study. Rats are housed in groups of 3-5 in large cages on ventilated shelves kept under standard animal care conditions—30-70% Humidity, 68-79° F. and 12 hr light/dark cycle under the supervision of IUCUC and an attending veterinarian. Rats are monitored daily for gross behavioral changes, such as lack of activity, lack of grooming, lack of eating/drinking, intolerability as a humane endpoint. Any rat demonstrating these behaviors are removed from the study upon recommendation from the attending veterinarian.

Study Design: The study design is depicted in Table 2 below. Rat pupils are dilated and protected from light. Then, they are sedated with ketamine/xylazine. A Micron IV small animal funduscope (Phoenix Research), took a fundus of the rat eye. A thermal laser of 540 nm wavelength connected to the Micron IV custom laser attachment will make 3 lesions per eye. A fundus is then recorded to ensure the formation of a bubble in the Bruch's membrane confirming the success of the laser injury. Laser injuries are bilateral with 3 lesions per eye or 6 lesions per rat.

On Day 3 and Day 10, following sedation, 5 μL of a bispecific antibody construct or vehicle will be administered intravitreally into using a 33G Hamilton syringe at the pars plana region. For fluorescein angiography at Day 22, rats are sedated with ketamine/xylazine and will be injected with 10% fluorescein sodium at 1 μL/g dose. Fundus images at an excitation wavelength of 488 nm are captured. Lesion quantification on the fundus images is done using an image analysis software, Photoshop CS Adobe Inc. Lesion size will be measured to calculate the neovascularization while the integrated density of fluorescein intensity will be measured to indicate vascular leak within the neovascular area. Overlapping or hemorrhaged lesions will be excluded from analysis. Upon euthanization, right eyes will be enucleated and fixed in Davidson's overnight at room temperature, then transferred to 70% EtOH at 4° C. until ready for analysis. Left eyes will be enucleated, dissected into retina and RPE/Choroid and snap frozen until ready for analysis.

Quantification of neovascularization in lesion area and vascular density in retina can be carried out by ImageJ, an open source software developed by the National Institutes of Health. P values can be assessed by Student's t test (significant change, p<0.05).

TABLE 2
Study Design

| Arm | Induction | Treatment | Treatment Details | Assessment |
|---|---|---|---|---|
| 1 | Bilateral Laser | Vehicle | Bilateral intravitreal | In-vivo fluorescein |
| | CNV | n = 8 | injection on Day 3 | angiography on Day 22; |
| 2 | 3 lesions/eye | Test Article #1 | 5 μl/eye | Terminal enucleation of |
| | Day 1 | n = 6 | | eyes with right eyes |
| 3 | | Test Article #2 | | processed for future |
| | | n = 6 | | histological analysis and |
| 4 | | Test Article #3 | | left eyes dissected to |
| | | n = 6 | | yield retina, and |
| 5 | | Test Article #4 | | RPE/choroid, separately, |
| | | n = 6 | | with tissue snap-frozen in |
| 6 | | Test Article #5 | | liquid N₂ |
| | | n = 6 | | |

### Example 8: Pharmacokinetic (PK) Studies of Tie2/VEGF Bispecifics in Adult Mice

This study will be done to compare the PK profile of the bispecific antibody constructs against a clinical control, Eylea® or a non-relevant IgG in whole eye or retinal or choroid tissue, and serum at various time points to assess durability.

Intravitreal Administration and Terminal Harvests: All animals will be treated in accordance with the Association for the Research in Vision and Ophthalmology Statement for the Use of Animals in Ophthalmic and Vision Research and will be approved by the Institutional Animal Care and Use Committee at UNITY Biotechnology.

Male C57BL/6J mice, 10⁻¹² weeks old are obtained from The Jackson Laboratory and allowed to acclimate for 48 hours prior to the start of the study. Intravitreal injections will be done using a Hamilton syringe attached to a Harvard apparatus pump at a flow rate of 12 μL/min. Following a small incision to be made about 1-2 mm behind the ora serrata, a 45° beveled needle (34G) will be inserted into the vitreal chamber. Upon sedation from isoflurane exposure, mice will be injected IVT 2PL with either Eylea® (Aflibercept) or the faricimab murine surrogate (“FMS”), or a non-relevant IgG (NSigG) or a bispecific antibody construct unilaterally (only right eye) at 10 μg/eye. For the duration experiment mice will be sacrificed at 2 h, 6 h, 1d, 2d, 3d, 5d, 7d and 14d after IVT administration. At these specific timepoints, the deeply anesthetized mouse is sacrificed by cervical dislocation. The thoracic cavity will be exposed, and blood will be collected from the lower heart chambers by cardiac puncture using a 25G needle into a serum separator tube and incubated for 30 min at 4° C. (BD Vacutainer™ Venous Blood Collection Tubes, Fisher Scientific). The right eye will be enucleated, the retina and the choroid dissected apart, snap frozen, and then stored at −80° C. Once incubated, blood will be centrifuged at 3000G for 10 min. The resulting serum will be aliquoted into microtubes then stored at −80° C.

Meso Scale Discovery MSD IgG Analysis: The PK analysis will be done using the Human/NHP IgG Kit (Meso Scale Discovery). Frozen eyes or eye tissue will be homogenized with MSD Tris Lysis Buffer (MSD) with a Precellys homogenizer in a 0.5 mL Precellys tube with ceramic beads (Bertin instruments). The sample will be centrifuged at 13,000G for 10 min. The supernatant is collected and incubated at 4° C. until the plate is run.

Serum is thawed to 4° C. A 1:10 dilution of the whole eye lysate will be prepared with the Diluent 100™ buffer provided with the kit. A 1:10000 dilution of the serum was prepared with Diluent 100™ as well. To prepare standards, a stock of 200,000 pg/mL of Eylea® or a bispecific antibody construct will be diluted serially 4-fold to generate an 8 point standard curve. Standards will be prepared in either undiluted or diluted whole eye lysate or eye tissue lysate or diluted serum lysate.

To streptavidin coated 96 well 4 spot plates, MSD Tris 5% (w/v) blocking buffer will be added and incubated on an orbital shaker at 600 rpm for 30 min. Once completed, the plates will be washed with Tris-MSD Wash buffer 3× followed by the addition of 25PL of standards, undiluted whole eye lysate, diluted whole eye lysate and diluted serum. The samples will be incubated on the orbital shaker at 600 rpm for 2 hours. After washing 3×, the plates will be incubated with 25 μL/well of SULPHO-TAG Anti-Hu/NHP IgG Antibody prepared 1× in Diluent 100™. The plates will be incubated on the orbital shaker at 600 rpm for 2 hours away from direct sunlight. Plates will be washed 3× for a final time. 150 μL of 2× Read Buffer T will be added to each well and then read on the MSD S-600 Instrument.

The electrochemiluminescence signal generated from each well will be analyzed using the Discovery Workbench™ software. The sample concentrations will be determined by a four-parameter fit logistic regression analysis based on the standard curve. From this, human IgG levels in whole eye, the retina, the choroid and serum will be graphed on GraphPad Prism® and the PK profiles of the bispecific antibody constructs against Eylea® or a non-relevant IgG control will be compared.

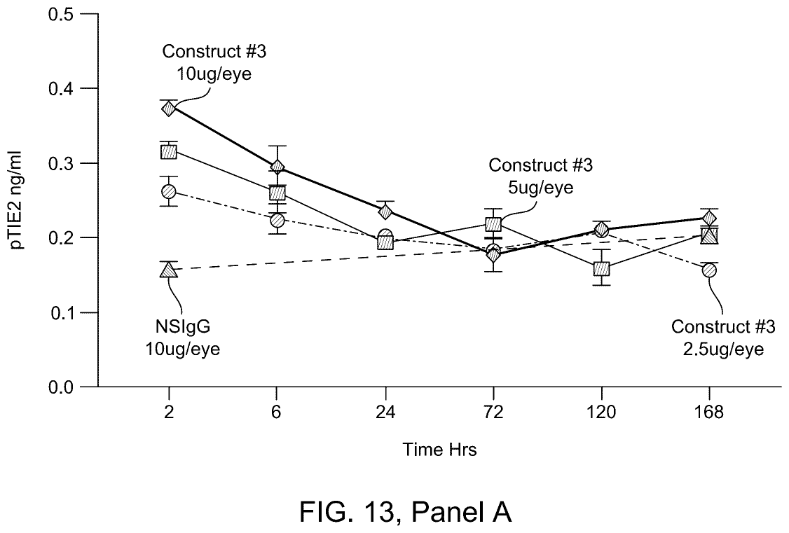

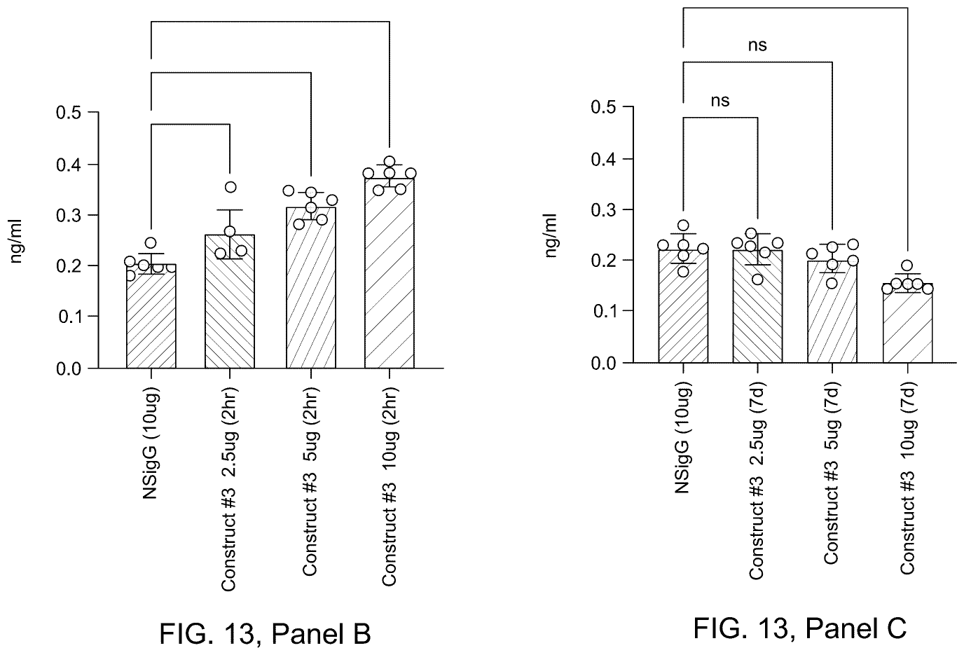

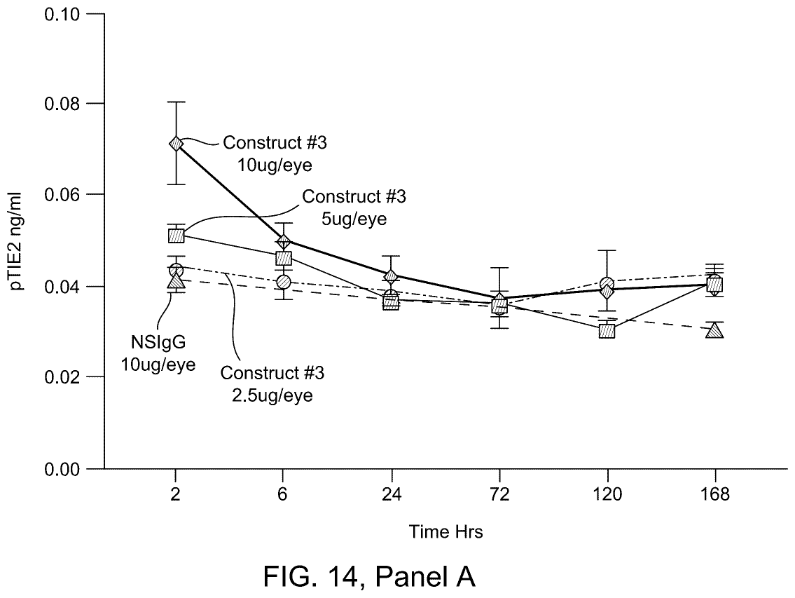

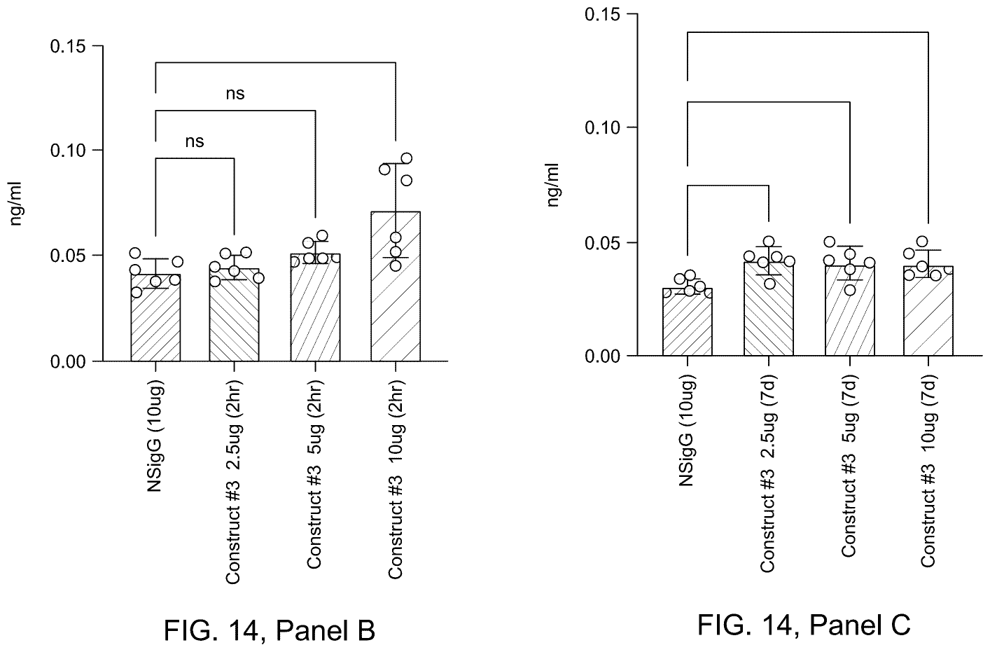

This experiment was performed with 2.5 ug/eye, 5 ug/eye or 10 ug/eye of either bispecific Construct #3 or a non-relevant IgG (NSigG) as control where target engagement was assessed on retina tissue or choroid tissue at timepoints 2 hrs, 6 hrs, 24 hrs, 72 hrs, 120 hrs, and 168 hrs or 7 days for Construct #3 and 2 hrs and 168 hrs or 7 days for an NSigG control. The results are shown in FIG. 13, panels A-C, for choroid tissue and FIG. 14, panels A-C, for retinal tissue. Construct #3 was able to engage target by activating the Tie2 pathway in choroid at 2 h and 6 hrs post IVT dosing in a dose-dependent manner. See FIG. 13, panels A and B. Similarly, Construct #3 was also able to engage target by activating the Tie2 pathway in retinal tissue at 2 hrs post IVT dosing in a dose-dependent manner. See FIG. 14, panels A and B.

### Example 9: ERK Pathway Activation by Western Blot

This study was to measure ERK pathway activation in HUVEC primary endothelial cells treated with a bispecific construct of the invention compared to the natural ligand Ang1, via a western blot method.

HUVEC cells from a single donor were used in the assay. The cells were cultured in endothelial cell growth media with supplements in a 37° C. incubator at 5% C02 and 3% oxygen. A day before the assay, cells were plated at 80,000 cells per well in a 96 well tissue culture plate. On the following day, cells were starved for 4 hrs in non-supplemental endothelial basal media. Post starvation of the cells, the non-supplemental endothelial basal media containing either the bispecific construct #3 or Ang1 control was added at three different concentrations—100, 25 and 6.25 nM to the cells. The cells were then incubated in a 37° C. incubator at 5% C02 and 3% oxygen for 20 min. After incubation, the media from the 96 well plates was removed, and the plates were stored in a −80° C. ultra-low freezer.

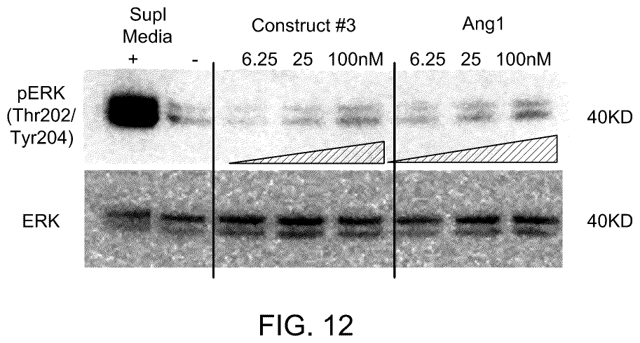

The HUVEC cells post treatment, were lysed in the 96 well plate using MSD TRIS lysis buffer. The lysis step was done by adding the lysis buffer at 50 μL per well and incubated the plate on a shaker at RT° for 1 hr. Post lysis of the cells, the lysates were collected and run on an SDS PAGE gel for western blot analysis. All the samples were assessed for total ERK/phospho-ERK. The lysates were run on a NuPAGE™ 4-12% gel (cat #NP0321 BOX). After electrophoresis, the gels were blotted onto nitrocellulose membrane and blocked for 30 min at RT° using 5% NFDM in TRIS-buffer saline. The blots were incubated in 1:1000 Primary antibody against ERK (cat #Rabbit mAb #4695, Cell Signaling Technology®), and pERK (cat #Rabbit mAb #3192, Cell Signaling Technology®) for 16 hours at 4° C. Post incubation the blots were washed and then incubated in secondary antibody—anti-rabbit antibody (cat #7074, Cell Signaling Technology®), for 2 hrs at RT°. The blots were exposed to substrate solution (cat #34095, ThermoFisher Scientific®). The results are shown in FIG. 12.

This experiment showed that for bispecific Construct #3, activation of the downstream ERK pathway was measured in a dose-dependent manner. See FIG. 12.

### Example 10: In Vivo Efficacy for Neovascularization in OIR Model

The OIR model was used to test the efficacy of bispecific constructs of the invention for neovascularization. Specifically, 78 mice were exposed to hyperoxia at the age of 7 days (P7) for 5 days. After that the mice were returned to normoxic conditions, and the treatment was administered via intraperitoneal (IP) injection at P12. Treatment consisted of 20 mg/kg of (a) Anti-Tie2 Clone #3 (n=11); (b) aflibercept-LALA-PG (n=11); (c) Construct #3 (n=10); (d) a positive control in aflibercept (Eyelea®) (n=10); as well as (e) 5 mg/kg of Construct #3 (n=10) and (f) a non-specific IgG negative control (NSIgG) (n=11). Five control mice were maintained under normoxic conditions.

Animal sacrifice and tissue collection was done at P17 and whole eyeballs were processed for retinal flat mounts and stained with Isolectin B4 to visualize blood vessels and neovascularization. Briefly, the flat mounts were washed with Tris-buffered saline (TBS) and blocked with 10% normal goat serum (NGS), 0.5% Triton X-100 in TBS pH 7.4 (TBST) for 1 hour in room temperature (RT). Samples were washed with TBS and incubated with fluorescein labeled Isolectin GS-1B4 (1:100, Invitrogen®) overnight at +4° C. in 1% NGS diluted in 0.1% TBST. Thereafter, the samples were washed 3×10 min with 1% NGS diluted in 0.1% TBST, counterstained with DAPI® (1:10 000, Sigma-Aldrich®) 30 minutes at RT in TBS and washed with TBS. Flat mounts were mounted with Fluoroshield™ mounting medium (Sigma-Aldrich®) on microscopic slides. Retinal flat mounts were imaged using a fluorescence microscope (Leica Thunder 3D Tissue Imager®, Leica Microsystems®). Avascular areas (AVAs) and neovascularization (NV) were quantified from the flat mounts with computational analysis which employed a proprietary algorithm owned by Experimentica® (Kuopio, Finland) which used a combination of convolutional neural network (CNN) designed for semantic segmentation and traditional computer vision algorithms. Quantitative data was graphed, analyzed, and presented as mean±standard deviation (SD). Statistical analyses were performed using the GraphPad®® Prism software (v9.2 GraphPad Software. USA). ROUT-method was performed to detect outliers in the data sets. Two-way RM ANOVA followed by S̆idák's multiple comparisons test was performed to the animal weight dat. One-Way ANOVA to the NV and AVA areas, followed by Dunnett's multiple comparisons test. Differences were considered statistically significant at the p<0.05 level.

Results: For avascular area reduction, the following was observed.

TABLE 3
Avascular Area Reduction Comparison

| | Concentration | Avascular Area |
|---|---|---|
| Test Article | (mg/kg) | Reduction (%) |
| Anti-Tie2 Clone #3 | 20 | 68% (p < 0.001) |
| aflibercept-LALA-PG | 20 | 24% (p < 0.001) |
| Construct #3 | 20 | 69% (p < 0.001) |
| Construct #3 | 5 | 27% (p < 0.05) |
| Aflibercept (Eyelea ®) | 20 | 59% (p < 0.001) |
| NSIgG | 20 | 0% (p < 0.001) |

Results: For neovascularization, the following was observed:

TABLE 4
Neovascularization Reduction Comparison

| | Concentration | Neovascularization |
|---|---|---|
| Test Article | (mg/kg) | Reduction (%) |
| Construct #3 | 20 | 36% (p < 0.001) |
| Aflibercept (Eyelea ®) | 20 | 75% (p < 0.001) |
| NSIgG | 20 | 0% (p < 0.001) |

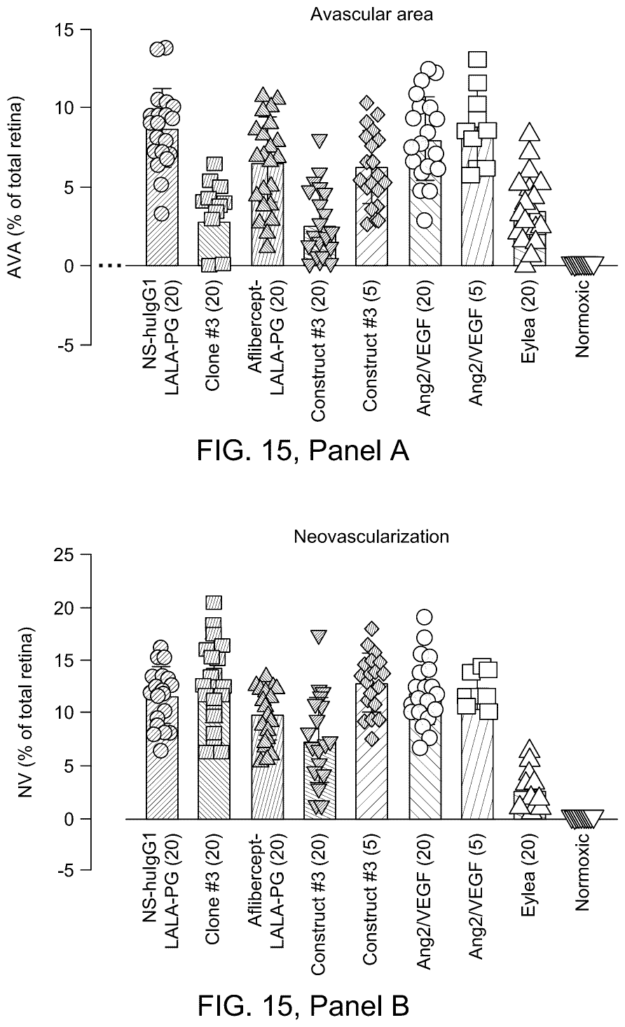

The OIR model performed as expected and the positive control, Eylea® (20 mg/kg), resulted in statistically significant reductions of both avascular and neovascular areas compared with the control group (NSIgG). Both avascular (FIG. 15, panel A and Table 3) and neovascular (FIG. 15, panel B and Table 4) areas were significantly reduced by Construct #3 (20 mg/kg). In addition to these, the Anti-Tie2 Clone #3 (20 mg/kg), aflibercept-LALA-PG (20 mg/kg) and Construct #3 (5 mg/kg) all reduced the size of avascular areas (FIG. 15, panel A and Table 3).

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. An isolated bispecific antibody that specifically binds to human Tie2 and to human VEGF selected from the group consisting of:
(a) an IgG heavy chain comprising an amino acid sequence of SEQ ID NO:4; and a light chain kappa comprising an amino acid sequence of SEQ ID NO:2;
(b) an IgG heavy chain comprising an amino acid sequence of SEQ ID NO:3; and a light chain kappa comprising an amino acid sequence of SEQ ID NO:2;
(c) an IgG heavy chain comprising an amino acid sequence of SEQ ID NO:1; and a light chain kappa comprising an amino acid sequence of SEQ ID NO:2;
(d) an IgG heavy chain comprising an amino acid sequence of SEQ ID NO:5; and a light chain kappa comprising an amino acid sequence of SEQ ID NO:2;
(e) an IgG heavy chain comprising an amino acid sequence of SEQ ID NO:6 and a light chain kappa comprising an amino acid sequence of SEQ ID NO:2;
(f) an amino acid sequence of SEQ ID NO:7;
(g) an amino acid sequence of SEQ ID NO:8;
(h) an amino acid sequence of SEQ ID NO:9;
(i) an amino acid sequence of SEQ ID NO:10;
(j) an IgG heavy chain comprising an amino acid sequence of SEQ ID NO:19; and a light chain kappa comprising an amino acid sequence of SEQ ID NO:2;
(k) an IgG heavy chain comprising an amino acid sequence of SEQ ID NO:20; and a light chain kappa comprising an amino acid sequence of SEQ ID NO:2;
(l) an IgG heavy chain comprising an amino acid sequence of SEQ ID NO:21; and a light chain kappa comprising an amino acid sequence of SEQ ID NO:2;
(m) an IgG heavy chain comprising an amino acid sequence of SEQ ID NO:22; and a light chain kappa comprising an amino acid sequence of SEQ ID NO:2;
(n) an IgG heavy chain comprising an amino acid sequence of SEQ ID NO:23; and a light chain kappa comprising an amino acid sequence of SEQ ID NO:2;
(o) an IgG heavy chain comprising an amino acid sequence of SEQ ID NO:24; and a light chain kappa comprising an amino acid sequence of SEQ ID NO:2;
(p) an IgG heavy chain comprising an amino acid sequence of SEQ ID NO:25; and a light chain kappa comprising an amino acid sequence of SEQ ID NO:2;
(q) an IgG heavy chain comprising an amino acid sequence of SEQ ID NO:26; and a light chain kappa comprising an amino acid sequence of SEQ ID NO:2;
(r) an IgG heavy chain comprising an amino acid sequence of SEQ ID NO:27; and a light chain kappa comprising an amino acid sequence of SEQ ID NO:2; and
(s) an IgG heavy chain comprising an amino acid sequence of SEQ ID NO:28; and a light chain kappa comprising an amino acid sequence of SEQ ID NO:2.

2. An isolated nucleic acid encoding the isolated bispecific antibody of claim 1.

3. A vector comprising the isolated nucleic acid of claim 2.

4. A host cell comprising the vector of claim 3.

5. A method of producing an isolated bispecific antibody, the method comprising culturing the host cell of claim 4 in a culture medium and isolating the resulting bispecific antibody.

6. An immunoconjugate comprising the isolated bispecific antibody of claim 1.

7. A fusion polypeptide comprising the isolated bispecific antibody of claim 1.

8. A pharmaceutical composition comprising the isolated bispecific antibody of claim 1, the immunoconjugate of claim 6, or the fusion polypeptide of claim 7.

9. A method of treating a disease in a subject in need thereof, the method comprising administering to the subject the pharmaceutical composition of claim 8.

10. The method of claim 9, wherein the disease comprises ocular diseases, infectious diseases, or ischemic diseases or injuries.

11. The method of claim 10, wherein the infectious diseases comprise sepsis, dengue virus infection, tuberculosis, coronavirus infection or influenza.

12. The method of claim 10, wherein the ischemic diseases comprise diabetic nephropathy, acute kidney injury, chronic kidney disease, organ transplantation, or critical limb ischemia.

13. The method of claim 10, wherein the ocular diseases comprise diabetic retinopathy (DR), diabetic macular edema (DME), non-proliferative diabetic retinopathy (nPDR), proliferative diabetic retinopathy (PDR), wet age-related macular degeneration (AMD), dry AMD, geographic atrophy (GA), retinopathy of prematurity (ROP), glaucoma, retinal vein occlusion (RVO), central RVO, or branched RVO.

14. The isolated bispecific antibody of claim 1, or the immunoconjugate of claim 6, or the fusion polypeptide of claim 7 for use in the treatment of the diseases of claims 10-13.

15. Use of the isolated bispecific antibody of claim 1, or the immunoconjugate of claim 6, or the fusion polypeptide of claim 7 for the manufacture of a medicament for treating the diseases of claims 10-13.
